(19) United States
(12) Patent Application Publication
(10) Pub. No.: US 2015/0374676 A1
(43) Pub. Date: Dec. 31, 2015

(54) HELICASE-PRIMASE INHIBITORS FOR USE IN A METHOD OF TREATING ALZHEIMER'S DISEASE

(71) Applicant: AICURIS GMBH & CO. KG, Wuppertal (DE)

(72) Inventor: Ruth ITZHAKI, Oxford (GB)

(73) Assignee: AICURIS GMBH & CO. KG, Wuppertal (DE)

(21) Appl. No.: 14/767,163

(22) PCT Filed: Feb. 12, 2014
(86) PCT No.: PCT/EP2014/052743

§ 371 Date: Aug. 11, 2015

(30) Foreign Application Priority Data
Feb. 12, 2013 (EP) 13154982.6

(51) Int. Cl. A61K 31/4439; A61K 31/185; A61K 9/14

(52) U.S. Cl. CPC A61K 31/4439; A61K 9/14; A61K 31/185

(57) ABSTRACT

The present invention relates to the use of helicase-primase inhibitors in a method of treating Alzheimer's Disease (AD). Particularly, the present invention relates to the use of helicase-primase inhibitors in a method of treating AD in a subject that is having HSV-1 infection and is having AD or is having HSV-1 infection and is suspected of having AD.

The provided antiviral helicase-primase inhibitors affect the accumulation of the key AD proteins amyloid beta and abnormally phosphorylated tau that occur during HSV-1 infection.

### FIELD OF THE INVENTION

The present invention relates to the field of neurodegenerative diseases, in particular to the field of Alzheimer's disease (hereinafter abbreviated AD).

Essentially, the present invention relates to the novel use of helicase-primase inhibitors (hereinafter abbreviated HPIs) in a method of treating AD.

Specifically, the present invention relates to the novel use of HPIs in a method of treating AD in a subject that is having herpes simplex virus type 1 (hereinafter abbreviated HSV-1) infection and is having AD or is having HSV-1 infection and is suspected of having AD.

Moreover, the present invention relates to the novel use of the specific crystalline mono mesylate monohydrate salt of the HPI N-[5-(aminosulfonyl)-4-methyl-1,3-thiazol-2-yl]-N-methyl-2-[4-(2-pyridinyl)phenyl]-acetamide in a method of treating AD in a subject that is having HSV-1 infection and is having AD or is having HSV-1 infection and is suspected of having AD.

Specifically, the present invention also relates to the novel use of the specific crystalline mono mesylate monohydrate salt of the HPI N-[5-(aminosulfonyl)-4-methyl-1,3-thiazol-2-yl]-N-methyl-2-[4-(2-pyridinyl)phenyl]-acetamide in a method of treating AD in a subject that is having HSV-1 infection and is having AD or is having HSV-1 infection and is suspected of having AD, whereby said crystalline mono mesylate monohydrate salt has a definite particle size range, particle size distribution and a specific surface area range, which has demonstrated increased long term stability and release kinetics from pharmaceutical compositions.

The present invention also relates to pharmaceutical compositions containing said crystalline N-[5-(aminosulfonyl)-4-methyl-1,3-thiazol-2-yl]-N-methyl-2-[4-(2-yridinyl)phenyl]acetamide mono mesylate monohydrate salt having the afore-mentioned particle size range, particle size distribution and specific surface area range.

Furthermore, the present invention relates to the pharmacokinetic (PK) and pharmacodynamic (PD) in vivo profiles of the free base form of N-[5-(aminosulfonyl)-4-methyl-1,3-thiazol-2-yl]-N-methyl-2-[4-(2-pyridinyl)phenyl]-acetamide resulting from crystalline N-[5-(aminosulfonyl)-4-methyl-1,3-thiazol-2-yl]-N-methyl-2-[4-(2-pyridinyl)phenyl]-acetamide mono mesylate monohydrate salt administration to a subject that is having HSV-1 infection and is having AD or is having HSV-1 infection and is suspected of having AD, whereby said mono mesylate monohydrate salt is administered in a pharmaceutical composition of the invention.

The resulting PK/PD profiles of the free base of N-[5-(aminosulfonyl)-4-methyl-1,3-thiazol-2-yl]-N-methyl-2-[4-(2-pyridinyl)phenyl]-acetamide are useful in methods of treating AD in a subject that is having HSV-1 infection and is having AD or is having HSV-1 infection and is suspected of having AD in accordance with the invention.

### BACKGROUND OF THE INVENTION

Neurodegenerative disease is the generic term for the progressive loss of function and structure of neurons as a result of clinically manifested neurodegenerative processes in a subject. Among the class of neurodegenerative diseases, AD affects over 26 million people world-wide, and the numbers will increase constantly (Brookmeyer R. et al., Forecasting the global burden of Alzheimer's disease. Alzheimer's and Dementia. 3(3):186-91 (2007)).

AD is characterized by loss of neurons and synapses in the cerebral cortex and in certain subcortical regions. This loss results in gross atrophy of the affected regions, including degeneration in the temporal lobe and parietal lobe, and parts of the frontal cortex and cingulate gyrus.

The causes of AD are not fully understood by the scientific community and current methods of treatment remain ineffective. Thus, AD represents a great health and social economical problem world-wide, which requires great efforts being made to develop further techniques and methods for early detection and effective methods of treatment of the AD types.

Generally, two main types of AD are yet known, namely early-onset AD (also commonly referred to as familial AD) and late-onset AD (also commonly referred to as sporadic AD):
a) Early-onset AD represents a rare form of AD in which people are diagnosed with the disease before the age of 65. Only less than 10% of all AD patients number among this type. Due to premature aging, people with Down syndrome are particularly at risk for early-onset AD, and are often in their mid- to late 40s or early 50s when symptoms first appear. Hereby, younger people suffering from said early-onset AD also show earlier brain abnormalities.
In this regard, familial AD represents a form of early-onset AD that is known to be entirely due to inherited causes. Familial AD appears to be linked with a genetic defect on one of three possible mutant genes, each located on three different chromosomes.
Family history is the second strongest risk factor for AD following advanced age. Twin and family studies indicated that genetic factors are estimated to play a role in at least 80% of AD cases. However, the inheritance of AD exhibits a dichotomous pattern. On the one hand, rare mutations in APP, PSEN1, and PSEN2 virtually guarantee early-onset familial AD, which represents about 5% of AD. On the other hand, common gene polymorphisms, such as the ε4 and ε2 variants of the apolipoprotein E gene (APOE) gene, can influence susceptibility for about 50% of the common late-onset AD. These four genes account for 30% to 50% of the inheritability of AD (Tanzi R. E., The Genetics of Alzheimer Disease, Cold Spring Harbor Perspectives in Medicine, pages 1-10; (2012)).
b) Late-onset AD represents the most common form of AD, accounting for about 90% of cases, and usually occurring after the age of 65. Late-onset AD strikes almost half of all people over the age of 85 and may or may not be due to hereditary causes.

It is further known that certain patients already suffering from mild cognitive impairment (hereinafter abbreviated MCI) may gradually develop the full clinical symptoms of a neurodegenerative disease such as the AD types described above. Therefore, MCI is known as a prodromal stage that may convert to AD (Schroeter M. L. et al., Neural Correlates of Alzheimer's Disease and Mild Cognitive Impairment: A Systematic and Quantitative Meta-Analysis involving 1,351 Patients. NeuroImage. 47(4):1196-1206. (2009) doi:10.1016/j.neuroimage.2009.05.037).

In more detail, AD is clinically characterized by a progressive memory loss and a decline of cognitive function. Histopathologically, AD is characterized by
a) extracellular deposition of fragments of accumulated amyloid beta precursor proteins, so called Aβ-peptides (hereinafter abbreviated Aβ), leading to the characteristic amyloid plaques (senile plaques) in brain of a subject suffering AD, and
b) intracellular deposits of abnormally phosphorylated tau proteins (hereinafter abbreviated P-tau), leading to the characteristic neurofibrillary tangles in brain of a subject suffering AD.

The Aβ-fragments are generated by subsequent cleavages by two aspartic proteases, BACE1 and presenilin 1, resulting in the liberation of Aβ-peptides of various lengths, namely Aβ 1-38/40/42.

There is evidence in the field of AD research that formation of aggregated Aβ-peptides, particularly Aβ 1-42 contributes to synaptic dysfunction and oxidative stress, which results in neuronal degeneration.

Recently, it could be further observed that HSV-1 infection is linked to AD. There is broad evidence that HSV-1 persists in a latent form inside nerve cells in brain of a subject. It has been proposed that re-activation and spreading of HSV-1 infections particularly contributes to cognitive decline that is may be associated with AD.

In more detail, it is known that HSV-1 is present (Jamieson G. A. et al., Latent herpes simplex virus type 1 in normal and Alzheimer's disease brains. J Med Virol 33, 224-227 (1991)) and can be active (Wozniak M. A. et al., Productive herpes simplex virus in brain of elderly normal subjects and Alzheimer's disease patients. J. Med. Virol. 75, 300-306 (2005)) in the brains of a high proportion of elderly people and it is a risk factor for AD when present in the brains of people who possess a specific genetic factor, namely the type 4 allele of the apolipoprotein E gene; APOE4 (Itzhaki R. F. et al., Herpes simplex virus type 1 in brain and risk of Alzheimer's disease. Lancet 349, 241-244 (1997)).

Furthermore, variation at the APOE locus may be associated with clinical manifestations of HSV-1 infection (Itzhaki et al., Herpes simplex virus type 1 in brain and risk of Alzheimer's disease. Lancet 349, 241-244 (1997); Koelle D. M. et al., APOE genotype is associated with oral herpetic lesions but not genital or oral herpes simplex virus shedding. Sex Transm Infect. 86(3): 202-206 (2010)).

In addition, there is sufficient evidence that the virus HSV-1 might be responsible for the abnormal protein deposits—amyloid plaques and neurofibrillary tangles—assumed to be central to AD pathogenesis. The essential findings in this regard can be summarized as follows:
(1) HSV-1 causes Aβ accumulation in the brains of infected mice (Wozniak M. A. et al., Herpes simplex virus infection causes cellular beta-amyloid accumulation and secretase upregulation. Neurosci Lett 429, 95-100 (2007)).
(2) in brains of AD patients, the HSV-1 DNA is located very specifically within amyloid plaques (Wozniak M. A. et al., Herpes simplex virus type 1 DNA is located within Alzheimer's disease amyloid plaques. J Pathol 217, 131-138 (2009b)).
(3) in cell cultures, HSV-1 causes production of the main components of amyloid plaques and neurofibrillary tangles, i.e. Aβ (Wozniak M. A. et al., Herpes simplex virus infection causes cellular beta-amyloid accumulation and secretase upregulation. Neurosci Lett 429(2-3), 95-100 (2007)); (Piacentini, R. et al., HSV-1 promotes Ca(2+)-mediated APP phosphorylation and Aβ accumulation in rat cortical neurons. Neurobiol Aging DOI: 10.1016/j.neurobiolaging.2010.06.009 (2010)); (Santana, S. et al., Herpes simplex virus type I induces the accumulation of intracellular beta-amyloid in autophagic compartments and the inhibition of the non-amyloidogenic pathway in human neuroblastoma cells. Neurobiol Aging doi:10.1016/j.neurobiolaging (2011)) and P-tau, respectively (Lerchundi R. et al., Tau Cleavage at D421 by Caspase-3 is Induced in Neurons and Astrocytes Infected with Herpes Simplex Virus Type 1. J Alzheimers Dis 23, 513-520 (2011)); (Wozniak M. A. et al., Alzheimer's disease-specific tau phosphorylation is induced by herpes simplex virus type 1. J Alzheimers Dis 16, 341-350 (2009a)); and (Zambrano A. et al., Neuronal cytoskeletal dynamic modification and neurodegeneration induced by infection with herpes simplex virus type 1. J Alzheimers Dis 14, 259-269 (2008)).

These findings support a potential role for HSV-1 in AD and suggested that acyclovir (hereinafter abbreviated ACV), a known antiviral agent, might be effective at slowing the progression of AD (Wozniak M. A. and Itzhaki R. F., Antiviral agents in Alzheimer's disease: hope for the future? Therap. Adv. in Neurol. Disorders; Review (2010)).

Cheng et al. (2011) investigated HSV-1 particles emerging from infected cells while observing under live confocal imaging of green fluorescent protein tagged to the HSV-1 within the investigated cells. Cheng et al. described the linkage between the amyloid precursor proteins and the HSV-1 particles, and they postulated an effect of this association on synaptic function (Cheng et al., Herpes Simplex Virus Dances with Amyloid Precursor Protein while Exiting the Cell. PLoS ONE, 6 (3): e17966 DOI: 10.1371/journal.pone.0017966 (2011)).

The above studies revealed that newly produced viral particles exit the cell nucleus and then bud into cellular membranes containing Aβ-peptides.

The interaction between viral particles and cellular Aβ-peptides results in changes in cellular architecture and the distribution of Aβ-peptides, the major component of the characteristic senile plaques to be found in the brains of patients suffering from AD. Results from said Cheng et al. (2011) study indicated that most intracellular HSV-1 particles undergo frequent, dynamic interplay with Aβ-peptides, which facilitates viral transport while interfering with normal Aβ-peptide transport and distribution. This dynamic interaction reveals a mechanism by which HSV-1 infection may lead to AD.

### Underlying Problem of the Invention

As stated above, current methods of treatment for patients suffering AD remain ineffective. The virus HSV-1 was found to influence the accumulation of the insoluble protein plaques derived from Aβ and P-tau, which are the key proteins involved in the neurodegenerative processes of AD.

ACV is an inhibitor of alpha herpes viruses such as HSV-1 and varicella zoster virus, and is used to combat a variety of disorders including cold sores, genital herpes, herpes simplex encephalitis and shingles.

ACV is a synthetic nucleoside analogue active against herpesviruses. Nucleoside analogues are molecules that act like nucleosides in DNA synthesis. They include a range of antiviral products used to prevent viral replication in infected cells. The most commonly used is ACV.

ACV is poorly water-soluble and has poor oral bioavailability (about 15-30%), hence intravenous administration is necessary if high concentrations are required. When orally administered, peak plasma concentrations occur after 1 to 2 hours. The elimination half-life (t_{1/2}) of ACV depends according to age group; neonates have a t_{1/2} of 4 hours, children of 1 to 12 years have a t_{1/2} of 2 to 3 hours; whereas adults have a t_{1/2} of 3 hours (Zovirax (acyclovir) dosing, indications, interactions, adverse effects, and more. Medscape Reference. WebMD. Retrieved 5 Feb. 2014).

Wozniak et al. (2011) examined the effect of ACV on the HSV-1 induced production of Aβ and P-tau, and they found that ACV, in fact, reduced the virus-induced accumulation of Aβ and P-tau in HSV-1-infected cell cultures (Wozniak M. A. et al., Antivirals reduce the formation of key Alzheimer's disease molecules in cell cultures acutely infected with herpes simplex virus type 1. PLoS One 6, e25152. (2011)).

However, nucleoside analogues as ACV for use in methods of treatment of AD do not completely abrogate HSV replication under treatment. As shown for genital herpes, there are still episodes of high titer shedding (>10⁴ HSV DNA copies) under therapy, which can lead to recurrences and even transmission of the infection (50% of cases) (Schiffer J T et al., Frequent release of low amounts of herpes simplex virus from neurons: results of a mathematical model. Sci Transl Med. 2009 Nov. 18; 1(7):7ra16).

This lack of activity can be attributed to two characteristics of the nucleoside analogues: first, the requirement to become activated in the cell by the viral thymidine kinase and second, their short half-life (Schiffer J T et al., Detailed analysis of mucosal herpes simplex virus-2 replication kinetics with and without antiviral therapy. J Antimicrob Chemother. 2011 November; 66(11):2593-600). Furthermore, usage of ACV in methods of treatment of AD may lead to development of resistant strains. Even though these are comparatively rare in immunocompetent subjects they can become more frequent in immunocompromised patients (Piret and Boivin, Resistance of herpes simplex viruses to nucleoside analogues: mechanisms, prevalence, and management. Antimicrob Agents Chemother. 2011 February; 55(2):459-72).

Moreover, ACV needs initial activation by viral thymidine kinases (Elion et al., Selectivity of action of an antiherpetic agent, 9-(2-hydroxyethoxymethyl)guanine; Proc. Natl. Acad. Sci., 74:5716-5720, (1977)). Therefore, ACV needs an initial HSV infection to become active.

### Solution to the Underlying Problem by the Invention

The present invention, surprisingly and unexpectedly, provides for HPIs according to the general Formula (I) for the novel use in methods of treatment of AD:

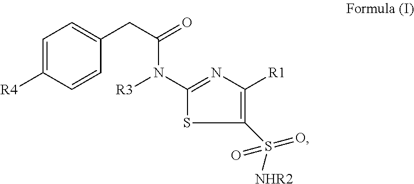

wherein
   R1 is selected from hydrogen, C1-C4 alkyl, or cycloalkyl,
   R2 is selected from hydrogen, C1-C4 alkyl, or cycloalkyl,
   R3 is selected from hydrogen, alkyl, cycloalkyl, heterocycloalkyl, haloalkyl, hydroxyalkyl, or alkoxyalkyl
   R4 is selected from substituted or unsubstituted heteroaryl, or aryl;
whereby
   a cycloalkyl group denotes a non-aromatic ring system containing three to eight carbon atoms, wherein one or more of the carbon atoms in the ring can be substituted by a group being O, S, SO, SO₂, N or NR′;
   R′ is independently H, haloalkyl, hydroxyalkyl, alkyl, cycloalkyl, aryl, or heteroaryl;
   an aryl group denotes an aromatic group having five to ten carbon atoms, which can optionally be substituted by one or more substituents R″;
   R″ is independently H, —CO₂R′, —CONHR′, —CO-alkyl, —CN, alkyl, alkoxy, —OH, —SH, cycloalkyl, heterocycloalkyl, halogen, haloalkyl, haloalkyloxy, hydroxyalkyl, heteroaryl, or aryl
   a heteroaryl group denotes a five- or six-membered heterocyclic group which contains at least one heteroatom selected from O, N, and S and may be fused to another aromatic ring.

In particular, the present invention provides for HPIs according to the general Formula (I) for the novel use in methods of treatment of AD in a subject that is having HSV-1 infection and is having AD or is having HSV-1 infection and is suspected of having AD.

Accordingly, the solution to the problem underlying the invention is the provision of HPIs that target another mode of action for inhibiting HSV-1 replication and thus inhibiting the accumulation of the AD key proteins Aβ and P-tau.

Moreover, the herein provided HPIs according to the general Formula (I) exhibit superior HSV-1 inhibitory efficacy, leading to effective reduction in Aβ and P-tau accumulation.

Thus, the HPIs of the present invention are suitable for use in methods of treatment of AD in a subject that is having HSV-1 infection and is having AD or that is having HSV-1 infection and is suspected of having AD.

The expression “suspected of having AD” denotes a subject or a patient group in accordance with the invention that is in the prodromal stage of MCI, and thus susceptible to progress (convert) to the clinical symptoms of AD. The person skilled in the art is aware that such conversion or progression from the prodromal stage of MCI to AD may be gradually.

The expression “helicase-primase inhibitor(s)” and its abbreviation “HPI(s)” with the context of the present invention denote the thiazolyl amide derivatives according to Formula (I) or pharmaceutically acceptable derivatives thereof, or a stereoisomer thereof as well as the respective pharmaceutically acceptable salts, solvates or hydrates thereof, which target a different stage of HSV-1 viral DNA replication, namely the helicase-primase complex. Accordingly, the HPIs according to Formula (I) do not need initial activation by viral thymidine kinases.

In this regard, thiazolyl amide derivatives, methods for their synthesis and its use in methods of treatment and/or for prevention of viral infection, particularly for the use in methods of treating infections caused by HSV have been described in WO 01/47904.

HPIs as drug candidates for the treatment of HSV infections have been described (Kleymann G. et al., New helicase-primase inhibitors as drug candidates for the treatment of herpes simplex disease. Nat Med 8, 392-398 (2002)).

Birkmann A. et al. (2011) summarized that the helicase-primase complex functions at the viral replication fork of HSV (Birkmann A. et al., Helicase-primase inhibitors as the potential next generation of highly active drugs against herpes simplex viruses, Future Virology, Vol. 6, No. 10, Pages 1199-1209, DOI 10.2217/fvl.11.28 (doi:10.2217/fvl.11.28) (2011)).

The term “helicase-primase complex” denotes a complex that unwinds the double-stranded HSV DNA and synthesises oligoribonucleotide primers for DNA synthesis by the viral DNA polymerase. This also pertains HSV-1 replication with the context of the present invention.

It should be emphasized that the HPIs of the invention—as sharply distinct compound class from known nucleoside analogues—surprisingly and unexpectedly exhibit superior inhibitory effects on formation of AD key proteins Aβ and P-tau (as shown in FIGS. 1 and 2); especially as the detailed underlying mechanism of HSV initiated Aβ and P-tau formation is not yet known in full.

The provided HPIs according to Formula (I) or pharmaceutically acceptable derivatives thereof, or a stereoisomer thereof as well as the respective pharmaceutically acceptable salts, solvates or hydrates thereof are capable to act as HSV-1 specific antiviral agents without the need for prior activation, and thus are superior agents for use in methods of treatment of AD in subjects having HSV-1 infection and AD or in subjects having HSV-1 infection and being suspected of having AD.

With the context of the present invention “act as HSV-1 specific antiviral agents without the need for prior activation” means that the HPIs of the invention also protect healthy, non-infected cells from becoming infected by HSV-1 since the HPIs of the invention are already antivirally active without an initial HSV infection. This means, due to the usage of the HPIs of the invention as anti-HSV-1 agents, non-infected cells remain unaffected and are thus prevented from HSV-1 infection, whereas nucleoside analogues in general, and thus also ACV, always need an initial HSV infection to become active.

Moreover, the provided HPIs according to general Formula (I) and their pharmaceutically active equivalents exhibit longer half-life as compared to ACV and therefore a lower risk of drug troughs (see Birkmann et al., Safety and human pharmacokinetics of AIC316, a potent helicase-primase inhibitor of herpes simplex virus (HSV), presented at 24^{th} International Conference on antiviral research, Sofia, Bulgaria—Abstracts/Antiviral Research 90 (2011) A21-A78).

The person skilled in the art can derive additional objects and advantages of the present invention from the following more detailed description.

### BRIEF DESCRIPTION OF THE INVENTION

The present invention relates to the HPIs according to general Formula (I) or pharmaceutically acceptable derivatives thereof, or a stereoisomer thereof for use in a method of treating AD:

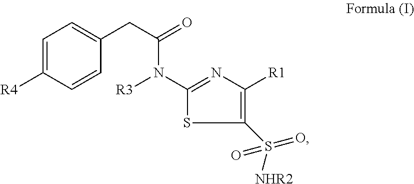

wherein
   R1 is selected from hydrogen, C1-C4 alkyl, or cycloalkyl,
   R2 is selected from hydrogen, C1-C4 alkyl, or cycloalkyl,
   R3 is selected from hydrogen, alkyl, cycloalkyl, heterocycloalkyl, haloalkyl, hydroxyalkyl, or alkoxyalkyl
   R4 is selected from substituted or unsubstituted heteroaryl, or aryl;
whereby
   a cycloalkyl group denotes a non-aromatic ring system containing three to eight carbon atoms, wherein one or more of the carbon atoms in the ring can be substituted by a group being O, S, SO, SO₂, N or NR′;
   R′ is independently H, haloalkyl, hydroxyalkyl, alkyl, cycloalkyl, aryl, or heteroaryl;
   an aryl group denotes an aromatic group having five to ten carbon atoms, which can optionally be substituted by one or more substituents R″;
   R″ is independently H, —CO₂R′, —CONHR′, —CO-alkyl, —CN, alkyl, alkoxy, —OH, —SH, cycloalkyl, heterocycloalkyl, halogen, haloalkyl, haloalkyloxy, hydroxyalkyl, heteroaryl, or aryl
   a heteroaryl group denotes a five- or six-membered heterocyclic group which contains at least one heteroatom selected from O, N, and S and may be fused to another aromatic ring.

Further the present invention relates to the pharmaceutically acceptable salts, solvates, and hydrates of the HPIs according to general Formula (I) for use in a method of treating AD.

In another embodiment, the present invention relates to the HPIs according to general Formula (I) or pharmaceutically acceptable derivatives thereof, or a stereoisomer thereof or pharmaceutically acceptable salts, solvates, and hydrates thereof for use in a method of treating AD in a subject that is having HSV-1 infection and is having AD or is having HSV-1 infection and is suspected of having AD.

It has been found by the inventors that the HPIs according to the general Formula (I) or pharmaceutically acceptable derivatives thereof, or a stereoisomer thereof as well as pharmaceutically acceptable salts, solvates and hydrates thereof are effective inhibitors of HSV-1 infections, and concomitant, surprisingly and unexpectedly, exhibit superior inhibitory efficacy for the accumulating key AD proteins Aβ and P-tau that occur during HSV-1 infection.

Thus, the present invention provides for a novel antiviral approach for a method of treating the neurodegenerative processes of AD. In particular, the present invention provides for a novel approach for a method of treating AD in a subject that is having HSV-1 infection and is having AD or is having HSV-1 infection and is suspected of having AD.

It is another objective of the present invention to also provide for a specific form of a salt of the HPI compound N-[5-(aminosulfonyl)-4-methyl-1,3-thiazol-2-yl]-N-methyl-2-[4-(2-pyridinyl)phenyl]acetamide which exhibits improved properties in regard to stability and bioavailability, and thus makes this specific form of the salt preferable for the manufacturing of pharmaceutical compositions and to provide pharmaceutical compositions containing such a specific salt so that these pharmaceutical compositions exhibit improved properties in regard to stability and bioavailability of the contained specific form of a salt of the compound N-[5-(aminosulfonyl)-4-methyl-1,3-thiazol-2-yl]-N-methyl-2-[4-(2-pyridinyl)phenyl]acetamide. Hereby, it should be noted that specifically in regard of the crystalline mono mesylate monohydrate salt of N-[5-(aminosulfonyl)-4-methyl-1,3-thiazol-2-yl]-N-methyl-2-[4-(2-pyridinyl)phenyl]-acetamide, only the free base form of N-[5-(aminosulfonyl)-4-methyl-1,3-thiazol-2-yl]-N-methyl-2-[4-(2-pyridinyl)phenyl]-acetamide exhibits bioavailability in a subject since the administered crystalline mono mesylate monohydrate salt of N-[5-(aminosulfonyl)-4-methyl-1,3-thiazol-2-yl]-N-methyl-2-[4-(2-pyridinyl)phenyl]-acetamide turns in vivo into the corresponding free base form.

Accordingly, another aspect of the present invention is directed to the PK profiles resulting from administration of the crystalline mono mesylate monohydrate salt of N-[5-(aminosulfonyl)-4-methyl-1,3-thiazol-2-yl]-N-methyl-2-[4-(2-pyridinyl)phenyl]-acetamide in vivo, when administered in a pharmaceutical composition of the instant invention to a subject that is having HSV-1 infection and is having AD or is having HSV-1 infection and is suspected of having AD. The PK in vivo profiles of the free base of N-[5-(aminosulfonyl)-4-methyl-1,3-thiazol-2-yl]-N-methyl-2-[4-(2-pyridinyl)phenyl]-acetamide enable sufficient absolute bioavailability of 70%±30% thereof.

The objectives of the present invention are solved by the teaching of the independent claims. Further advantageous features, aspects, and details of the invention are evident from the dependent claims, the description, the drawings (Figs.), and the examples of the present application.

### DETAILED DESCRIPTION OF THE INVENTION

The subject matter of the present invention relates to the novel use of HPIs in a method of treating AD. In particular, the present invention relates to the novel use of HPIs in a method of treating AD in a subject that is having HSV-1 infection and is having AD or said subject is having HSV-1 infection and is suspected of having AD, while affecting the accumulation of the key AD proteins Aβ and P-tau through targeting the helicase-primase complex of HSV-1.

The expressions “is having HSV-1 infection” and “is HSV-1 positive” with the context of the present invention denote a subject or a patient group that is positive for HSV-1 infection when clinically examined by a HSV-test suitable to identify HSV-1 infection ex vivo.

The expressions “is having AD” and “is suspected of having AD” with the context of the present invention denote a subject or a patient group that is positive for AD when clinically examined by a person skilled in the art and found positive for the manifested dementia symptoms of having difficulties with many areas of mental function, including:
emotional behavior or personality
language
memory
perception
thinking and judgment (cognitive skills)
forgetfulness.

In this regard, early symptoms of AD can include:
difficulties for the subject performing tasks that take some thought, but used to come easily, such as balancing a checkbook, playing complex games (such as bridge), and learning new information or routines
getting lost on familiar routes
language problems, such as trouble finding the name of familiar objects
losing interest in things previously enjoyed, flat mood
misplacing items
personality changes and loss of social skills.

As the AD becomes worse, symptoms are more obvious and interfere with the subject's ability to take care of them. Symptoms can include:
change in sleep patterns, often waking up at night
delusions, depression, agitation
difficulty doing basic tasks, such as preparing meals, choosing proper clothing, and driving
difficulty reading or writing
forgetting details about current events
forgetting events in your own life history, losing awareness of who you are
hallucinations, arguments, striking out, and violent behavior
poor judgment and loss of ability to recognize danger
using the wrong word, mispronouncing words, speaking in confusing sentences
withdrawing from social contact

Subjects with severe AD can no longer:
understand language
recognize family members
perform basic activities of daily living, such as eating, dressing, and bathing.

In addition, the expressions “is having AD” and “is suspected of having AD” with the context of the present invention also denote a subject or a patient group that is positive for AD when clinically examined by a laboratory test suitable for diagnosing AD, those suitable being either an ex vivo PSEN1 test, and/or an ex vivo Tau/Aβ42 test, and/or the ex vivo APOE genotyping test as further set out below.

The person skilled in the art who is a clinician/physician, a medical biochemist, or trained medical staff is further aware of suitable tests for both in combination, meaning testing for HSV-1 infection in an ex vivo sample and testing for AD in an ex vivo sample of a subject for sake of evaluating and identifying a subject or a patient grouped that is addressed by the present invention.

### Identification of the Herein Addressed Subject/Patient Group

The subject/patient group addressed by the present invention are individuals that either
a) show at least the above-mentioned symptoms of early onset of AD, or the full symptoms of AD and severe AD, and/or are
b) positive for HSV-1 infection due to a clinical diagnosis and/or by a laboratory HSV-test as further set out below, and/or
c) show at least the below outlined signs of mild cognitive impairment (MCI), which may represents the prodromal stage of AD.
   Identification of a Subject being Positive for HSV-1 Infection

The person skilled in the art is aware that generally HSV infection causes distinct medical conditions, which can be diagnosed from the appearance of the subject. Common infection of the skin or mucosa potentially affects the face and the mouth (orofacial herpes), genitalia (genital herpes), or hands (herpetic whitlow). More serious disorders occur when the virus infects and damages the eye (herpes keratitis), or invades the central nervous system, damaging the brain (herpes encephalitis).

In all cases HSV is never removed from the body by the immune system. Following a primary infection, the virus enters the nerves at the site of primary infection, migrates to the cell body of the neuron, and becomes latent in the ganglion (cf Roizman et al. Herpes Simplex Viruses. In: Knipe D M, Howley P M, eds. Fields Virology. 5th ed. Lippincott Williams & Wilkins, 2502-2601 (2006)).

As a result of primary infection, the body produces antibodies to the particular type of HSV involved, preventing a subsequent infection of that type at a different site. Particularly in HSV-1 infected individuals, seroconversion after an oral infection will prevent additional HSV-1 infections such as whitlow, genital herpes, and herpes of the eye. Prior HSV-1 seroconversion seems to reduce the symptoms of a later HSV-2 infection, although HSV-2 can still be contracted.

Therefore, HSV-tests for the identification of HSV-1 infection in a subject that is having ADor is suspected of having AD in accordance with the present invention are suitable to discriminate HSV-1 infection from HSV-2 infection, and are selected from the group comprising HSV polymerase chain reaction (PCR) test, or HSV antigen detection test, or HSV-1 IgM rapid test, or HSV-1 IgG rapid test. The person skilled in the art knows these tests and how to utilize it for reliable discrimination between HSV-1 and HSV-2 infection.

The exemplary tests that are may be used for the identification of HSV-1 infection in a subject are described below in more detail:
Herpes virus antigen detection test: Cells from a fresh sore are scraped off and then smeared onto a microscope slide. This test finds markers (called antigens) on the surface of cells infected with the herpes virus. This test may be done with or in place of a viral culture.
HSV PCR test: A PCR test can be done on cells or fluid from a sore or on blood or on other fluid, such as spinal fluid. PCR finds the genetic material (DNA) of the HSV virus itself. This test is capable to discriminate between HSV-1 and HSV-2 infection. The PCR test is not often done on skin sores, but it is best for testing spinal fluid, for those rare cases in which herpes may cause an infection in or around the brain.
HSV-1 IgM and the HSV-1 IgG rapid test: The detection of HSV-1 IgM antibody enables effective diagnosis of acute or recent HSV-1 infection. The presence of HSV-1 IgG antibody in serum is an indication of previous exposure. A significant increase in HSV-1 IgG is an indication of reactivation, current or recent infection. Herpes Simplex Virus (HSV-1) IgM test cassette and HSV-1 IgG test cassette are two types of serum test devices, which are used to respectively detect the IgM and IgG antibodies in human serum or plasma samples. These two HSV-1 screening tests are intended to screen the samples of the subject.

To reliably identify patients suffering from HSV-1 infection the Immunodot glycoprotein G-specific (IgG) HSV test is more than 98% specific at discriminating HSV-1 from HSV-2 infection (cf. Ashley R L, et. al., “Comparison of Western blot (immunoblot) and glycoprotein G-specific immunodot enzyme assay for detecting antibodies to herpes simplex virus types 1 and 2 in human sera”. J. Clin. Microbiol. 26 (4): 662-7. PMC 266403. PMID 2835389, (1998).

Accordingly, the expression “clinically examined by HSV-test” with the context of the present invention denotes the performance of any of the above-mentioned HSV-tests to identify subjects having HSV-1 infection.

### Identification of Subjects Having MCI

To identify subjects that exhibit at least signs of MCI, and that are thus under suspect of having AD the following symptoms should be manifested in said subject during clinical examination by a person skilled in the art (based on M. S. Albert et al., The diagnosis of mild cognitive impairment due to Alzheimer's disease: Recommendations from the National Institute on Aging and Alzheimer's Association workgroup, Alzheimer's & Dementia (2011) 1-10):

Symptoms of a subject having MCI include:
difficulty performing more than one task at a time
difficulty solving problems
forgetting recent events or conversations
taking longer to perform more difficult activities

Therefore, in a preferred embodiment in accordance with the invention the subject(s) intended for administration of the herein provided HPIs for use in a method of treating AD shows at least the below manifested symptoms of mild cognitive impairment during clinical examination, i.e.
difficulty performing more than one task at a time
difficulty solving problems
forgetting recent events or conversations
taking longer to perform more difficult activities.

Accordingly, in another embodiment the present invention provides for HPIs according to Formula (I) for use in a method of treating AD in a subject that is having HSV-1 infection and is having AD or is having HSV-1 infection and is suspected of having AD, wherein said subject shows at least the below manifested symptoms of mild cognitive impairment during clinical examination, i.e.
a change in cognition
impairment in one or more cognitive domains
preservation of independence in functional abilities
not demented,
   and said subject is positive for HSV-1 infection when clinically examined by HSV-test.

Further criteria for the diagnosis of MCI are as follows:

### Concern Regarding a Change in Cognition

There should be evidence of concern about a change in cognition, in comparison to the subject's prior level. This concern can be obtained from the subject, from an informant who knows the subject well, or from a skilled clinician observing the subject.

### Impairment in One or More Cognitive Domains

There should be evidence of lower performance in one or more cognitive domains that is greater than would be expected for the subject's age and educational background. If repeated assessments are available, then a decline in performance should be evident over time. This change can occur in a variety of cognitive domains, including: memory, executive function, attention, language and visuospatial skills. An impairment in episodic memory (i.e., the ability to learn and retain new information) is seen most commonly in MCI patients who subsequently progress to a diagnosis of AD (so-called AD converter).

### Preservation of Independence in Functional Abilities

Subjects with MCI commonly have mild problems performing complex functional tasks they used to be able to perform, such as paying bills, preparing a meal, shopping at the store. They may take more time, be less efficient, and make more errors at performing such activities than in the past. Nevertheless, they generally maintain their independence of function in daily life, with minimal aids or assistance.

### Not Demented

These cognitive changes should range from mild to significant impairment in social or occupational functioning. It should be emphasized that the diagnosis of MCI requires evidence of intra-individual change. If an individual has only been evaluated once, change will need to be inferred from the history and/or evidence that cognitive performance is impaired beyond what would have been expected for that individual. Serial evaluations are optimal.

In a further specific embodiment the present invention provides for HPIs according to general Formula (I) or pharmaceutically acceptable derivatives thereof, or a stereoisomer thereof as well as pharmaceutically acceptable salts, solvates and hydrates thereof for use in a method of treating AD in a subject that is having HSV-1 and is having AD or is having HSV-1 and is suspected of having AD, characterized in that said subject is positive in an ex vivo HSV-test as outlined above and possesses a specific genetic factor, namely the type 4 allele of the apolipoprotein E gene (APOE4) when said subject is positive for APOE4 in an ex vivo venous blood sample examined by APOE genotyping test known to the person skilled in the art.

Thus, the inventors have implicated HSV-1 infection in AD for therapeutic approaches, discovering that it confers a strong risk for manifestation of AD in individuals who also carry a specific genetic factor, namely the type 4 allele of the APOE gene when positive for APOE4 in an APOE genotyping test.

With this context the expression “specific genetic factor” means a polymorphism present in said gene that is known to be linked to a causative role for AD, e.g. the ε4 variant of the APOE gene.

In a further specific embodiment the present invention provides for HPIs according to general Formula (I) or pharmaceutically acceptable derivatives thereof, or a stereoisomer thereof as well as pharmaceutically acceptable salts, solvates and hydrates thereof for use in a method of treating AD in a subject that is having HSV-1 and is having AD or is having HSV-1 and is suspected of having AD, characterized in that said subject is positive for HSV-1 infection in an ex vivo HSV-test and said subject is positive for PSEN1 in an ex vivo PSEN1 test known to the person skilled in the art.

“PSEN1” stands for Presenilin-1 (PS-1) that is a protein in humans, which is encoded by the PSEN1 gene. Presenilin 1 is one of the four core proteins of the presenilin complex, which mediate the proteolytic events of different proteins in the cell, including gamma secretase. The person skilled in the art is aware that gama-secretase is considered to play a very important role in generation of Aβ and its accumulation, which is related to the onset of AD from the beta-amyloid precursor protein and thus a reliable indicator for developing or manifested AD in a subject. The person skilled in the art is aware that a subject being positive in a PSEN1 test is positive for having AD or is suspected of having AD.

In another specific embodiment the present invention provides for HPIs according to general Formula (I) or pharmaceutically acceptable derivatives thereof, or a stereoisomer thereof as well as pharmaceutically acceptable salts, solvates and hydrates thereof for use in a method of treating AD in a subject that is having HSV-1 and is having AD or is having HSV-1 and is suspected of having AD, characterized in that said subject is positive for HSV-1 infection in an ex vivo HSV-test and said subject is positive for the presence of Aβ42 and P-tau in an ex vivo Tau/Aβ42 test known to the person skilled in the art.

In this regard, the person skilled in the art is aware that a positive ex vivo Tau/Aβ42 test reflects the presence of Aβ42 and P-tau in a subject that is thus positive for having AD or suspected of having AD.

As stated above, HSV-1 infection is linked with the two main neuropathological features of AD, namely the senile plaques and the neurofibrillary tangles.

Specifically, the inventors found that targeting the accumulation of Aβ and/or P-tau via the HPIs according to Formula (I) of the instant invention, ameliorates AD in a subject that is having HSV-1 and is having AD or is having HSV-1 and is suspected of having AD.

Thus, in a preferred embodiment the present invention relates to the use of the HPIs according to general Formula (I) as set out below, or pharmaceutically acceptable derivatives thereof, or a stereoisomer thereof in a method of treating AD:

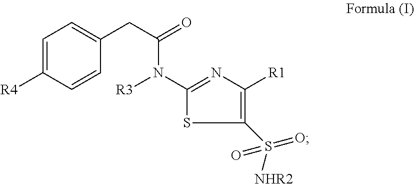

wherein
   R1 is selected from hydrogen, C1-C4 alkyl, or cycloalkyl, and/or
   R2 is selected from hydrogen, C1-C4 alkyl, or cycloalkyl, and/or
   R3 is selected from hydrogen, alkyl, cycloalkyl, heterocycloalkyl, haloalkyl, hydroxyalkyl, or alkoxyalkyl and/or
   R4 is selected from substituted or unsubstituted heteroaryl, or aryl;
whereby
   a cycloalkyl group denotes a non-aromatic ring system containing three to eight carbon atoms, wherein one or more of the carbon atoms in the ring can be substituted by a group being O, S, SO, SO₂, N or NR′;
   R′ is independently H, haloalkyl, hydroxyalkyl, alkyl, cycloalkyl, aryl, or heteroaryl;
   an aryl group denotes an aromatic group having five to ten carbon atoms, which can optionally be substituted by one or more substituents R″;
   R″ is independently H, —CO₂R′, —CONHR′, —CO-alkyl, —CN, alkyl, alkoxy, —OH, —SH, cycloalkyl, heterocycloalkyl, halogen, haloalkyl, haloalkyloxy, hydroxyalkyl, heteroaryl, or aryl
a heteroaryl group denotes a five- or six-membered heterocyclic group which contains at least one heteroatom selected from O, N, and S and may be fused to another aromatic ring.

In another embodiment, the present invention provides for the HPIs according to general Formula (I) as set out below or pharmaceutically acceptable derivatives thereof, or a stereoisomer thereof for use in a method of treating AD in a subject that is having HSV-1 infection and is having AD or is having HSV-1 infection and is suspected of having AD:

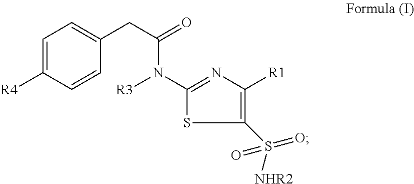

wherein
   R1 is selected from hydrogen, C1-C4 alkyl, or cycloalkyl, and/or
   R2 is selected from hydrogen, C1-C4 alkyl, or cycloalkyl, and/or
   R3 is selected from hydrogen, alkyl, cycloalkyl, heterocycloalkyl, haloalkyl, hydroxyalkyl, or alkoxyalkyl, and/or
   R4 is selected from substituted or unsubstituted heteroaryl, or aryl;
whereby
   a cycloalkyl group denotes a non-aromatic ring system containing three to eight carbon atoms, wherein one or more of the carbon atoms in the ring can be substituted by a group being O, S, SO, SO₂, N or NR′;
   R′ is independently H, haloalkyl, hydroxyalkyl, alkyl, cycloalkyl, aryl, or heteroaryl;
   an aryl group denotes an aromatic group having five to ten carbon atoms, which can optionally be substituted by one or more substituents R″;
   R″ is independently H, —CO₂R′, —CONHR′, —CO-alkyl, —CN, alkyl, alkoxy, —OH, —SH, cycloalkyl, heterocycloalkyl, halogen, haloalkyl, haloalkyloxy, hydroxyalkyl, heteroaryl, or aryl
   a heteroaryl group denotes a five- or six-membered heterocyclic group which contains at least one heteroatom selected from O, N, and S and may be fused to another aromatic ring.

Further, in a specific embodiment the present invention relates to the use of pharmaceutically acceptable salts, solvates, and hydrates of the compounds according to general Formula (I) in a method of treating AD.

In another specific embodiment the present invention relates to the use of pharmaceutically acceptable salts, solvates, and hydrates of the compounds according to general Formula (I) in a method of treating AD in a subject that is having HSV-1 infection and is having AD or is having HSV-1 infection and is suspected of having AD.

Surprisingly, it was found by the inventors that the specific crystalline mono mesylate monohydrate salt of N-[5-(aminosulfonyl)-4-methyl-1,3-thiazol-2-yl]-N-methyl-2-[4-(2-pyridinyl)phenyl]acetamide exhibits advantageous PK/PD in vivo profiles of the resultant free base of N-[5-(aminosulfonyl)-4-methyl-1,3-thiazol-2-yl]-N-methyl-2-[4-(2-pyridinyl)phenyl]acetamide, if said crystalline mono mesylate monohydrate salt has a specific PSD (particle size distribution), PSR (particle size range) and SSA (specific surface area). The resultant free base of N-[5-(aminosulfonyl)-4-methyl-1,3-thiazol-2-yl]-N-methyl-2-[4-(2-pyridinyl)phenyl]acetamide is the pharmacologic active component resulting from administration of the crystalline mono mesylate monohydrate salt of N-[5-(aminosulfonyl)-4-methyl-1,3-thiazol-2-yl]-N-methyl-2-[4-(2-pyridinyl)phenyl]acet-amide.

Thus, the present invention also relates to the crystalline N-[5-(aminosulfonyl)-4-methyl-1,3-thiazol-2-yl]-N-methyl-2-[4-(2-pyridinyl)-phenyl]acetamide mono methanesulfonic acid monohydrate of the following formula

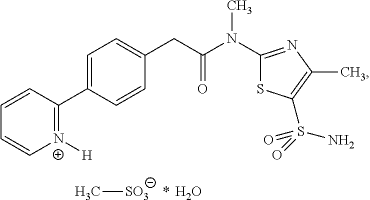

for use in a method of treating AD in a subject that is having HSV-1 infection and is having AD or is having HSV-1 infection and is suspected of having AD; wherein the crystalline N-[5-(aminosulfonyl)-4-methyl-1,3-thiazol-2-yl]-N-methyl-2-[4-(2-pyridinyl)-phenyl]acetamide mono methanesulfonic acid monohydrate particles have a particle size range from 1 to 500 μm, a particle size distribution which is defined by d(0.1) from 2 to 100 μm, d(0.5) from 30 to 210 μm and d(0.9) from 70 to 400 μm and a specific surface area of less than 1.0 m²/g.

In a preferred embodiment the particle size of the crystalline N-[5-(amino-sulfonyl)-4-methyl-1,3-thiazol-2-yl]-N-methyl-2-[4-(2-pyridinyl)-phenyl]acetamide mesylate monohydrate particles is within the range of 1 μm to 500 μm, preferably in the range of 1.5 μm to 450 μm and more preferably in the range of 2 μm to 400 μm. Thus, the particle size range (PSR) of the mesylate monohydrate is from 1.0 μm to 500 μm, preferably from 1.5 μm to 450 μm, more preferably from 2.0 μm to 400 μm, still more preferably from 2.5 μm to 300 μm and most preferably from 3.0 μm to 250 μm. If the PSR is not mentioned at all or if reference to the PSR is made without stating a definite value, it shall be referred to a particle size range from 1 to 500 μm.

In another preferred embodiment the particle size distribution of the crystalline N-[5-(amino-sulfonyl)-4-methyl-1,3-thiazol-2-yl]-N-methyl-2-[4-(2-pyridinyl)-phenyl]acet-amide mono mesylate monohydrate particles is characterized by d(0.1) from 4 to 100 μm, d(0.5) from 30 to 210 μm and d(0.9) from 70 to 400 μm, more preferably d(0.1) from 6 to 95 μm, d(0.5) from 50 to 200 μm and d(0.9) from 100 to 390 μm, still more preferably d(0.1) from 7 to 90 μm, d(0.5) from 70 to 190 μm and d(0.9) from 130 to 380 μm, still more preferably d(0.1) from 8 to 85 μm, d(0.5) from 80 to 185 μm and d(0.9) from 160 to 370 μm, still more preferably d(0.1) from 9 to 80 μm, d(0.5) from 90 to 180 μm and d(0.9) from 180 to 360 μm, still more preferably d(0.1) from 10 to 75 μm, d(0.5) from 100 to 175 μm and d(0.9) from 200 to 350 μm and most preferably d(0.1) from 11 to 70 μm, d(0.5) from 110 to 170 μm and d(0.9) from 220 to 340 μm.

Furthermore, in another preferred embodiment the specific surface area of the crystalline particles is less than 1.0 m²/g, more preferably less than 0.9 m²/g, still more preferably less than 0.8 m²/g, still more preferably less than 0.7 m²/g, still more preferably less than 0.6 m²/g, still more preferably less than 0.5 m²/g, still more preferably less than 0.4 m²/g and most preferably the SSA of the particles is less than 0.3 m²/g.

In a certain aspect of the present invention said specific surface area is typically greater than about 0.01 to 0.06 m²/g, the lower limit not being particularly important.

Accordingly, in another aspect of the invention the specific surface area is within a range of 0.01 to 0.99 m²/g, preferably within a range of 0.05 to 0.99 m²/g, even more preferably within a range of 0.06 to 0.99 m²/g, most preferred within a range of 0.06 to 0.29 m²/g.

As used herein the terms “mono mesylate monohydrate”, or “crystalline mono mesylate monohydrate”, or “mono methanesulfonic acid monohydrate”, or “crystalline mono methanesulfonic acid monohydrate”, or “crystalline N-[5-(amino-sulfonyl)-4-methyl-1,3-thiazol-2-yl]-N-methyl-2-[4-(2-pyridinyl)-phenyl]acetamide mono mesylate monohydrate”, or “N-[5-(amino-sulfonyl)-4-methyl-1,3-thiazol-2-yl]-N-methyl-2-[4-(2-pyridinyl)-phenyl]acetamide mono methanesulfonic acid monohydrate” refers to the crystalline N-[5-(amino-sulfonyl)-4-methyl-1,3-thiazol-2-yl]-N-methyl-2-[4-(2-pyridinyl)-phenyl]-acetamide mono methanesulfonic acid monohydrate having the PSD, PSR and SSA as defined herein. Thus, these terms ever denote the specific mono mesylate monohydrate salt in accordance with the invention, whereas the term “free base of N-[5-(amino-sulfonyl)-4-methyl-1,3-thiazol-2-yl]-N-methyl-2-[4-(2-pyridinyl)-phenyl]acetamide”, “free base form”, and “free base” ever denote the free base form of N-[5-(amino-sulfonyl)-4-methyl-1,3-thiazol-2-yl]-N-methyl-2-[4-(2-pyridinyl)-phenyl]acetamide, which also is ever the pharmacologically active form of N-[5-(amino-sulfonyl)-4-methyl-1,3-thiazol-2-yl]-N-methyl-2-[4-(2-pyridinyl)-phenyl]acetamide in the human body.

The crystalline N-[5-(amino-sulfonyl)-4-methyl-1,3-thiazol-2-yl]-N-methyl-2-[4-(2-pyridinyl)-phenyl]acetamide mono mesylate monohydrate obtainable according to the above disclosed synthesis is then used to prepare a pharmaceutical composition thereof, wherein the crystalline N-[5-(amino-sulfonyl)-4-methyl-1,3-thiazol-2-yl]-N-methyl-2-[4-(2-pyridinyl)phenyl]acetamide mono mesylate monohydrate has the particle size distribution (PSD), specific surface area (SSA) and particle size range (PSR) as defined herein.

Due to various possible particle size ranges, particle size distributions and specific surface areas adjustable by various techniques, it was unforeseeable for the skilled person that the ranges for PSD, SSA and PSR as defined herein are the most suitable for preparing pharmaceutical compositions. Particularly, it was unforeseeable for the skilled person that the ranges for PSD, SSA and PSR of the crystalline mono mesylate monohydrate salt of N-[5-(aminosulfonyl)-4-methyl-1,3-thiazol-2-yl]-N-methyl-2-[4-(2-pyridinyl)-phenyl]acetamide as defined herein lead to advantageous PK/PD in vivo profiles of its pharmacologically active free base form as exemplarily depicted in the FIGS. 3 to 7. Said Figures show exemplarily PK/PD in vivo profiles of the resultant free base form in vivo, when administered orally either as single dose or in the form of multiple dosages as crystalline mono mesylate monohydrate salt of N-[5-(aminosulfonyl)-4-methyl-1,3-thiazol-2-yl]-N-methyl-2-[4-(2-pyridinyl)-phenyl]acetamide.

A further advantage of the tablets comprising the specific crystalline mono mesylate monohydrate salt of N-[5-(aminosulfonyl)-4-methyl-1,3-thiazol-2-yl]-N-methyl-2-[4-(2-pyridinyl)-phenyl]acetamide according to the invention is that the tablet will have an optimized dissolution rate based on its particle size distribution of the crystalline form of N-[5-(aminosulfonyl)-4-methyl-1,3-thiazol-2-yl]-N-methyl-2-[4-(2-pyridinyl)-phenyl]acetamide mono methanesulfonic acid monohydrate which has the PSD, PSR and SSA as defined herein and thus, the drug may be absorbed into the blood stream much faster compared to the free base form of N-[5-(aminosulfonyl)-4-methyl-1,3-thiazol-2-yl]-N-methyl-2-[4-(2-pyridinyl)-phenyl]acetamide or other salts thereof as API in a tablet. Furthermore, the surprising dispersion times obtained with tablets according to the invention are advantageous for swallowable tablets. In a further embodiment, the tablets according to the invention can be presented for dispersion in water.

In respect of the stated above, the person skilled in the art understands that the dissolution behavior may be directly linked to resultant bioavailability properties of an active pharmaceutical ingredient in vivo. Accordingly, a high degree of absolute bioavailability may be expected based on the dissolution properties of the crystalline N-[5-(aminosulfonyl)-4-methyl-1,3-thiazol-2-yl]-N-methyl-2-[4-(2-pyridinyl)-phenyl]acetamide mono methanesulfonic acid monohydrate in a tablet of the invention.

Accordingly, the present invention, surprisingly und unexpectedly, also provides for chemically stable, orally administrable pharmaceutical compositions of the crystalline N-[5-(aminosulfonyl)-4-methyl-1,3-thiazol-2-yl]-N-methyl-2-[4-(2-pyridinyl)-phenyl]acetamide mono methanesulfonic acid monohydrate having PSD, PSR and SSA as defined herein, characterized by an absolute bioavailability of the resultant free base form of N-[5-(aminosulfonyl)-4-methyl-1,3-thiazol-2-yl]-N-methyl-2-[4-(2-pyridinyl)-phenyl]acetamide of at least 40 to 90%, preferably 50 to 90%, more preferably 60 to 85%, when administered in a pharmaceutical composition of the invention.

In yet another aspect the present invention, surprisingly und unexpectedly, also provides for chemically stable, orally administrable pharmaceutical compositions of the crystalline N-[5-(aminosulfonyl)-4-methyl-1,3-thiazol-2-yl]-N-methyl-2-[4-(2-pyridinyl)-phenyl]acetamide mono methanesulfonic acid monohydrate having PSD, PSR and SSA as defined herein, characterized by absolute bioavailability of the resultant free base form of N-[5-(aminosulfonyl)-4-methyl-1,3-thiazol-2-yl]-N-methyl-2-[4-(2-pyridinyl)-phenyl]acetamide of >40%, preferably >50%, even more preferably >70%, most preferred >80%, utmost preferred >90%, when administered in a pharmaceutical composition of the invention.

In yet another aspect the present invention provides for pharmaceutical compositions as described herein, effective to achieve an absolute bioavailability of 70%±30% of the resultant free base form of N-[5-(aminosulfonyl)-4-methyl-1,3-thiazol-2-yl]-N-methyl-2-[4-(2-pyridinyl)-phenyl]acetamide, when administered as crystalline N-[5-(aminosulfonyl)-4-methyl-1,3-thiazol-2-yl]-N-methyl-2-[4-(2-pyridinyl)-phenyl]acetamide mono methanesulfonic acid monohydrate particles having PSD, PSR and SSA as defined herein in a pharmaceutical composition containing at least 5 mg, preferably at least 10 mg, more preferably at least 20 mg, most preferred at least 25 mg thereof.

In yet another aspect of the invention said absolute bioavailability of 70%±30% of the resultant free base form of N-[5-(aminosulfonyl)-4-methyl-1,3-thiazol-2-yl]-N-methyl-2-[4-(2-pyridinyl)-phenyl]acetamide is achieved in a human.

In the context of the present invention the term “bioavailability” denotes a subcategory of absorption. Bioavailability denotes the fraction of an administered oral dose of the crystalline N-[5-(aminosulfonyl)-4-methyl-1,3-thiazol-2-yl]-N-methyl-2-[4-(2-pyridinyl)-phenyl]acetamide mono methanesulfonic acid monohydrate of the invention that reaches the systemic circulation of a subject as the resultant free base form of N-[5-(aminosulfonyl)-4-methyl-1,3-thiazol-2-yl]-N-methyl-2-[4-(2-pyridinyl)-phenyl]acetamide. By definition, when a medication is administered intravenously, its bioavailability is 100%. However, when a medication is administered via other routes (such as orally), its bioavailability generally decreases (due to incomplete absorption and first-pass metabolism) or may vary from individual to individual. Bioavailability is one of the essential tools in pharmacokinetics, as bioavailability must be considered when calculating dosages for non-intravenous routes of administration.

The crystalline mono mesylate monohydrate salt of N-[5-(aminosulfonyl)-4-methyl-1,3-thiazol-2-yl]-N-methyl-2-[4-(2-pyridinyl)phenyl]acetamide, wherein the PSR, PSD and SSA is as defined herein, exhibits increased long term stability properties and a desired release kinetic and long term stability from pharmaceutical compositions, which is superior to other salts of N-[5-(aminosulfonyl)-4-methyl-1,3-thiazol-2-yl]-N-methyl-2-[4-(2-pyridinyl)phenyl]acetamide, which are known in the state of the art including also other mesylate salts.

The inventive mono mesylate monohydrate salt is thus useful in a method of treatment of AD in a subject that is having HSV-1 infection and is having AD or is having HSV-1 infection and is suspected of having AD.

A preferred pharmaceutical composition according to the invention preferably comprises 5 to 70% by weight more preferably 10 to 30% by weight crystalline N-[5-(aminosulfonyl)-4-methyl-1,3-thiazol-2-yl]-N-methyl-2-[4-(2-pyridinyl)-phenyl]acet-amide mono methanesulfonic acid monohydrate (all percentage data are percentages by weight based on the weight of the pharmaceutical preparation), wherein the PSD, PSR and SSA is as defined herein. The pharmaceutical composition comprises usually 2 to 600 mg of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, preferably 5 to 500 mg, more preferably 10 to 300 mg and particularly preferably 20 to 200 mg, wherein PSD, PSR and SSA is as disclosed above.

A specifically preferred pharmaceutical composition of the invention comprises:

5%-30% crystalline N-[5-(aminosulfonyl)-4-methyl-1,3-thiazol-2-yl]-N-methyl-2-[4-(2-pyridinyl)phenyl]acetamide mono methanesulfonic acid monohydrate, wherein the PSD, PSR and SSA is as defined herein and preferably the particle size distribution is defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g, 5%-10% croscarmellose-sodium, 0.5 to 0.7% magnesium stearate, 40%-70% microcrystalline cellulose, 10%-20% mannitol and 0.5% to 1% colloidal anhydrous silica.

Another specifically preferred pharmaceutical composition according to the invention preferably comprise 30 to 90% more preferably 50 to 70% by weight crystalline N-[5-(aminosulfonyl)-4-methyl-1,3-thiazol-2-yl]-N-methyl-2-[4-(2-pyridinyl)-phenyl]acet-amide mono methanesulfonic acid monohydrate, wherein the PSD, PSR and SSA is as defined herein and preferably the particle size distribution is defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g (all percentage data are percentages by weight based on the weight of the pharmaceutical preparations). The pharmaceutical composition comprises usually 20 to 750 mg as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, wherein the PSD, PSR and SSA is as defined herein and preferably 50 to 500 mg as free base equivalent and particularly preferably 50 to 250 mg as free base equivalent based on a single dosage.

As used herein for the specifically given mg-dosages of the crystalline N-[5-(aminosulfonyl)-4-methyl-1,3-thiazol-2-yl]-N-methyl-2-[4-(2-pyridinyl)-phenyl]acetamide mono methanesulfonic acid monohydrate as API in a pharmaceutical composition of the invention, particularly for tablet formulations thereof are ever described as the free base equivalent dosage, which means that the content of crystalline N-[5-(aminosulfonyl)-4-methyl-1,3-thiazol-2-yl]-N-methyl-2-[4-(2-pyridinyl)-phenyl]acetamide mono methanesulfonic acid monohydrate is approximately 1.3 times higher as indicated. This is due to the fact that the pharmacologically active form in vivo is the free base form of N-[5-(aminosulfonyl)-4-methyl-1,3-thiazol-2-yl]-N-methyl-2-[4-(2-pyridinyl)-phenyl]acetamide, which is however administered as the mono mesylate monohydrate salt form having the characteristic PSD, PSR and SSA of the invention.

Therefore, the term “free base equivalent” as used herein and in the claims with the context of crystalline N-[5-(aminosulfonyl)-4-methyl-1,3-thiazol-2-yl]-N-methyl-2-[4-(2-pyridinyl)-phenyl]acetamide mono methanesulfonic acid monohydrate denotes the dosage of the pharmacologically active form, thus calculated as free base form of N-[5-(aminosulfonyl)-4-methyl-1,3-thiazol-2-yl]-N-methyl-2-[4-(2-pyridinyl)-phenyl]acetamide.

Particularly, the definite crystalline mono mesylate monohydrate salt of N-[5-(aminosulfonyl)-4-methyl-1,3-thiazol-2-yl]-N-methyl-2-[4-(2-pyridinyl)-phenyl]acetamide with the characteristic PSR, PSD and SSA as disclosed herein exhibits characteristic PK/PD profiles in vivo as the free base form when administered in a pharmaceutical composition in accordance with the invention.

Exposure of the free base form of N-[5-(aminosulfonyl)-4-methyl-1,3-thiazol-2-yl]-N-methyl-2-[4-(2-pyridinyl)-phenyl]acetamide to the human body may be measured by high pressure liquid chromatography (HPLC) by looking at different pharmacokinetic parameters in suitable bodily fluids such as for instance blood plasma and urine, the most common parameters being the Cₘₐₓ, the so-called area under the curve (AUC), and the terminal half-life (t_{1/2Z}). Hereto, the person skilled in the art understands that said parameters are determined by using adequate bioanalytical methods with adequate sensitivity, specificity, ruggedness, stability and repeatability, as for instance a qualified liquid chromatography triple quad mass spectrometry based method coupled with a suitable extraction method for the separation of the analyte from, e.g. the blood plasma. For example, AUC values may be calculated from 0-24 hours using the trapezoid method.

For instance, after administration of crystalline mono mesylate monohydrate salt of N-[5-(aminosulfonyl)-4-methyl-1,3-thiazol-2-yl]-N-methyl-2-[4-(2-pyridinyl)phenyl]-acetamide, its concentration in the blood increases in the form of the free base of N-[5-(aminosulfonyl)-4-methyl-1,3-thiazol-2-yl]-N-methyl-2-[4-(2-pyridinyl)phenyl]-acetamide until it reaches a peak concentration, which measured in blood by a suitable HPLC method is the Cₘₐₓ and the time taken to reach the Cₘₐₓ is termed tₘₐₓ. The area under the blood plasma concentration curve (area under the curve abbreviated as AUC) is another useful measurement and represents the drug exposure of the free base in the systemic circulation over a period of time; e.g. 0-24 h or 0-∞.

The mean Cₘₐₓ values are derived from averaging the highest observed free base concentration of N-[5-(aminosulfonyl)-4-methyl-1,3-thiazol-2-yl]-N-methyl-2-[4-(2-pyridinyl)phenyl]-acetamide for all members of a subject group under investigation.

The mean C_{max,ss} values are derived from averaging the highest observed free base concentration at steady state of N-[5-(aminosulfonyl)-4-methyl-1,3-thiazol-2-yl]-N-methyl-2-[4-(2-pyridinyl)phenyl]-acetamide for all members of a subject group under investigation.

In a specific aspect the present invention provides for a pharmaceutical composition as described above for the crystalline mono mesylate monohydrate salt of N-[5-(aminosulfonyl)-4-methyl-1,3-thiazol-2-yl]-N-methyl-2-[4-(2-pyridinyl)-phenyl]acetamide, effective to achieve a mean maximum blood plasma concentration (mean Cₘₐₓ) of the free base of N-[5-(aminosulfonyl)-4-methyl-1,3-thiazol-2-yl]-N-methyl-2-[4-(2-pyridinyl)-phenyl]acetamide in a subject of at least one of
a) 608±184 ng/ml for a 40 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
b) 1306±125 ng/ml for a 80 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
c) 2613±1341 ng/ml for a 160 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
d) 3600±752 ng/ml for a 240 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
e) 4648±1813 ng/ml for a 320 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
f) 6926±1656 ng/ml for a 400 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
g) 6921±2190 ng/ml for a 480 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered.

In yet another specific aspect the present invention provides for a pharmaceutical composition as described above for the crystalline mono mesylate monohydrate salt of N-[5-(aminosulfonyl)-4-methyl-1,3-thiazol-2-yl]-N-methyl-2-[4-(2-pyridinyl)-phenyl]acetamide, effective to achieve a mean maximum blood plasma concentration (mean Cₘₐₓ) of the free base of N-[5-(aminosulfonyl)-4-methyl-1,3-thiazol-2-yl]-N-methyl-2-[4-(2-pyridinyl)-phenyl] in a subject of at least one of
a) 608±184 ng/ml for a 40 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or effective to achieve an AUC₀₋₂₄ₕ of 10090±3114 ng-h/ml in a subject for a 40 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 72±3 h on average; said dosage being a single oral dose administered;
b) 1306±125 ng/ml for a 80 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or effective to achieve an AUC₀₋₂₄ₕ of 21940±2057 ng-h/ml in a subject for a 80 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 74±5 h on average; said dosage being a single oral dose administered;
c) 2613±1341 ng/ml for a 160 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or effective to a achieve an AUC₀₋₂₄ₕ of 40470±16700 ng-h/ml in a subject for a 160 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 63±6 h on average; said dosage being a single oral dose administered;
d) 3600±752 ng/ml for a 240 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or effective to achieve an AUC₀₋₂₄ₕ of 59610±12770 ng-h/ml in a subject for a 240 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 64±5 h on average; said dosage being a single oral dose administered;
e) 4648±1813 ng/ml for a 320 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or effective to achieve an AUC₀₋₂₄ₕ of 76250±27630 ng-h/ml in a subject for a 320 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 57±3 h on average; said dosage being a single oral dose administered;
f) 6926±1656 ng/ml for a 400 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or effective to achieve an AUC₀₋₂₄ₕ of 104800±25740 ng-h/ml in a subject for a 400 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 57±4 h on average; said dosage being a single oral dose administered;
g) 6921±2190 ng/ml for a 480 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or effective to achieve an AUC₀₋₂₄ₕ of 112800±34260 ng-h/ml in a subject for a 480 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 53±4 h on average; said dosage being a single oral dose administered.

In yet another specific aspect the present invention provides for a pharmaceutical composition as described above for the crystalline mono mesylate monohydrate salt of N-[5-(aminosulfonyl)-4-methyl-1,3-thiazol-2-yl]-N-methyl-2-[4-(2-pyridinyl)-phenyl]acetamide, effective to achieve a mean maximum blood plasma concentration at steady state (mean C_{max,ss}) of the free base of N-[5-(aminosulfonyl)-4-methyl-1,3-thiazol-2-yl]-N-methyl-2-[4-(2-pyridinyl)-phenyl]acetamide in a subject of at least one of
a) 1358±167 ng/ml for a 25 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a steady state dose achieved after once daily single doses administered for 21 days;
b) 6358±1701 ng/ml for a 100 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a steady state dose achieved after once daily single doses administered for 21 days;
c) 9987±2608 ng/ml for a 200 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a steady state dose achieved after once daily single doses administered for 21 days.

In yet another specific aspect the present invention provides for a pharmaceutical composition as described above for the crystalline mono mesylate monohydrate salt of N-[5-(aminosulfonyl)-4-methyl-1,3-thiazol-2-yl]-N-methyl-2-[4-(2-pyridinyl)-phenyl]acetamide, effective to achieve a mean maximum blood plasma concentration at steady state (mean C_{max,ss}) of the free base of N-[5-(aminosulfonyl)-4-methyl-1,3-thiazol-2-yl]-N-methyl-2-[4-(2-pyridinyl)-phenyl]acetamide in a subject of at least one of
a) 1358±167 ng/ml for a 25 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or effective to achieve an AUC_{τ,ss} of 23430±3020 ng-h/ml in a subject for a 25 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 69±6 h on average, said dosage being a steady state dose achieved after once daily single doses administered for 21 days;
b) 6358±1701 ng/ml for a 100 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or effective to achieve an AUC_{τ,ss} of 108800±28610 ng-h/ml in a subject for a 100 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 60±4 h on average, said dosage being a steady state dose achieved after once daily single doses administered for 21 days;
c) 9987±2608 ng/ml for a 200 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or effective to achieve an AUC_{τ,ss} of 168500±37970 ng-h/ml in a subject for a 200 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 57.19±5.451 h on average, said dosage being a steady state dose achieved after once daily single doses administered for 21 days.

In yet another specific aspect the present invention provides for a method of treatment of AD in a subject that is having HSV-1 infection and is having AD or is having HSV-1 infection and is suspected of having AD, wherein a mean maximum blood plasma concentration (mean Cₘₐₓ) of the free base of N-[5-(aminosulfonyl)-4-methyl-1,3-thiazol-2-yl]-N-methyl-2-[4-(2-pyridinyl)-phenyl]acetamide of at least one of
a) 608±184 ng/ml for a 40 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ₕ of 10090±3114 ng-h/ml for a 40 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, wherein t_{1/2z} is 72±3 h on average;
b) 1306±125 ng/ml for a 80 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ of 21940±2057 ng-h/ml for a 80 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, wherein t_{1/2z} is 74±5 h on average;
c) 2613±1341 ng/ml for a 160 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ of 40470±16700 ng-h/ml for a 160 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, wherein tinz is 63±6 h on average;
d) 3600±752 ng/ml for a 240 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ₕ of 59610±12770 ng-h/ml for a 240 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, wherein t_{1/2z} is 64±5 h on average;
e) 4648±1813 ng/ml for a 320 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ₕ of 76250±27630 ng-h/ml for a 320 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, wherein t_{1/2z} is 57±3 h on average;
f) 6926±1656 ng/ml for a 400 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ₕ of 104800±25740 ng-h/ml for a 400 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, wherein t_{1/2z} is 57±4 h on average;
g) 6921±2190 ng/ml for a 480 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ₕ of 112800±34260 ng-h/ml for a 480 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, wherein t_{1/2z} is 53±4 h on average,
is achieved in a human; and wherein said dosage is a single oral dose administered.

In yet another specific aspect the present invention provides for a method of treatment of AD in a subject that is having HSV-1 infection and is having AD or is having HSV-1 infection and is suspected of having AD, wherein a mean maximum blood plasma concentration at steady state (mean C_{max,ss}) of the free base of N-[5-(aminosulfonyl)-4-methyl-1,3-thiazol-2-yl]-N-methyl-2-[4-(2-pyridinyl)-phenyl]acetamide of at least one of
a) 1358±167 ng/ml for a 25 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC_{τ,ss} of 23430±3020 ng-h/ml for a 25 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, wherein t_{1/2z} is 69±6 h on average;
b) 6358±1701 ng/ml for a 100 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC_{τ,ss} of 108800±28610 ng-h/ml in a subject for a 100 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, wherein t_{1/2z} is 60±4 h on average;
c) 9987±2608 ng/ml for a 200 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or effective to achieve an AUC_{τ,ss} of 168500±37970 ng-h/ml in a subject for a 200 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 57.19±5.451 h on average,
is achieved in a human; and wherein said dosage is a steady state dose achieved after once daily single doses administered for 21 days.

In yet another specific aspect of the invention said mean maximum blood plasma concentration (mean Cₘₐₓ) of the free base of N-[5-(aminosulfonyl)-4-methyl-1,3-thiazol-2-yl]-N-methyl-2-[4-(2-pyridinyl)-phenyl]acetamide is achieved in a human.

In yet another specific aspect of the invention said mean maximum blood plasma concentration at steady state (mean C_{max,ss}) of the free base of N-[5-(aminosulfonyl)-4-methyl-1,3-thiazol-2-yl]-N-methyl-2-[4-(2-pyridinyl)-phenyl]acetamide is achieved in a human.

In yet another specific aspect of the invention said AUC₀₋₂₄ₕ, AUC_{0-∞} and t_{1/2z} is achieved in a human.

In yet another specific aspect of the invention said AUC_{τ,ss} and t_{1/2z} is achieved in a human.

As used in the specification and the claims “AUC_{τ,ss}” denotes the area under the analyte versus time concentration curve over a dosing interval (tau) at steady-state (ss), calculated by linear up/log down summation.

As used in the specification, the general expression “AUCₜ₁₋ₜ₂” denotes the area under the analyte versus time concentration curve from point in time t₁ to point in time t₂, calculated by linear up/log down summation. For example AUC₀₋₂₄ denotes the area under the analyte versus time concentration curve from point in time of administration (t₁=0) to the point in time of 24 h after administration (t₂=24 h). Accordingly, AUC_{0-∞} denotes the concentration from time of administration up to infinity, calculated as

$${AUC}_{0 - ∞} = {AUC}_{0 - last} + \frac{{C}_{last}}{{λ}_{z}} ,$$

wherein AUC₀₋ₗₐₛₜ is defined as the area under the analyte vs. time concentration up to time of the last qualifiable concentration, calculated by linear up/log down summation and Cₗₐₛₜ is defined as last quantifiable observed analyte concentration. λ_{z} is the apparent terminal elimination rate constant, determined by linear regression of terminal points of In-linear analyte concentration-time curve.

Cₘₐₓ is the maximal observed analyte concentration and tₘₐₓ is the time to reach Cₘₐₓ; t_{1/2z} is defined as the apparent terminal elimination half-life, calculated as

$${t}_{1 / 2 z} = \frac{ln ( 2 )}{{λ}_{z}} ,$$

wherein λ_{z} is defined as above.

As used in the specification and the claims “t_{1/2z}” denotes the apparent terminal elimination half-life calculated as: t_{1/2z}=0.693/λ_{z}. Thereby, λ_{z} denotes the apparent terminal elimination rate constant.

Further, it should be noted that the crystalline mono mesylate monohydrate salt of N-[5-(aminosulfonyl)-4-methyl-1,3-thiazol-2-yl]-N-methyl-2-[4-(2-pyridinyl)phenyl]-acetamide of the invention is used as API for tablet formulation in accordance with the invention, whereas the free base form of N-[5-(aminosulfonyl)-4-methyl-1,3-thiazol-2-yl]-N-methyl-2-[4-(2-pyridinyl)phenyl]-acetamide, having the formula

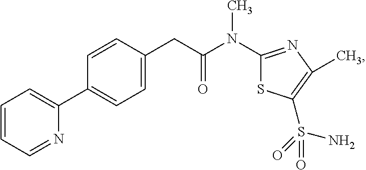 [SMILES: Cc1nc(N(C)C(=O)Cc2ccc(-c3ccccn3)cc2)sc1S(N)(=O)=O]

is the resultant pharmacologically active form in the body of a subject after administration of crystalline mono mesylate monohydrate salt of N-[5-(aminosulfonyl)-4-methyl-1,3-thiazol-2-yl]-N-methyl-2-[4-(2-pyridinyl)phenyl]-acetamide, preferably after oral administration thereof.

Further, the person skilled in the art understands that the pharmaceutical compositions of the invention among each other comprise physical or chemical dosage form characteristics, which may modulate either one of said mean Cₘₐₓ, AUC₀₋₂₄ₕ, AUC_{τ,ss}, AUC_{0-∞}, and t_{1/2z} as given in the above specific aspects of the invention.

Further, in accordance with the invention the person skilled in the art understands that food intake prior to administration of the crystalline mono mesylate monohydrate salt of N-[5-(aminosulfonyl)-4-methyl-1,3-thiazol-2-yl]-N-methyl-2-[4-(2-pyridinyl)phenyl]-acetamide of a subject may influence positively the in vivo PK/PD profile of the resultant free base of N-[5-(aminosulfonyl)-4-methyl-1,3-thiazol-2-yl]-N-methyl-2-[4-(2-pyridinyl)phenyl]-acetamide. Thus, in accordance with the invention a decreased absorption rate and a delayed tₘₐₓ of the resultant free base of N-[5-(aminosulfonyl)-4-methyl-1,3-thiazol-2-yl]-N-methyl-2-[4-(2-pyridinyl)phenyl]-acetamide may be expected in fasted subjects after administration of crystalline mono mesylate monohydrate salt of N-[5-(aminosulfonyl)-4-methyl-1,3-thiazol-2-yl]-N-methyl-2-[4-(2-pyridinyl)phenyl]-acetamide.

By contrast, food intake prior to administration of crystalline mono mesylate monohydrate salt of N-[5-(aminosulfonyl)-4-methyl-1,3-thiazol-2-yl]-N-methyl-2-[4-(2-pyridinyl)phenyl]-acetamide may lead to an increase in mean Cₘₐₓ of at least about 25% and an increase in AUC of at least about 10% of the resultant free base of N-[5-(aminosulfonyl)-4-methyl-1,3-thiazol-2-yl]-N-methyl-2-[4-(2-pyridinyl)phenyl]-acetamide in human blood plasma when measured by suitable HPLC method. Thereby t_{1/2z} remains constant.

In accordance with the invention, the administration of crystalline mono mesylate monohydrate salt of N-[5-(aminosulfonyl)-4-methyl-1,3-thiazol-2-yl]-N-methyl-2-[4-(2-pyridinyl)phenyl]-acetamide with the free base equivalent dosages as disclosed herein is safe and well tolerated by a subject in need thereof. No dose-dependent adverse events are to be expected when crystalline mono mesylate monohydrate salt of N-[5-(aminosulfonyl)-4-methyl-1,3-thiazol-2-yl]-N-methyl-2-[4-(2-pyridinyl)phenyl]-acetamide is administered as disclosed herein.

Further detailed information on methods of synthesis of crystalline mono mesylate monohydrate salt of N-[5-(aminosulfonyl)-4-methyl-1,3-thiazol-2-yl]-N-methyl-2-[4-(2-pyridinyl)phenyl]-acetamide, its general properties and other relevant background information can be derived from WO 2013/045491.

In another preferred embodiment, the present invention relates to the use of the HPIs according to general Formula (I), or pharmaceutically acceptable derivatives thereof, or a stereoisomer thereof as well as to pharmaceutically acceptable salts, solvates, and hydrates thereof, in a method of treating AD:
wherein
   R1 is selected from hydrogen, or C1-C4 alkyl, and/or
   R2 is selected from hydrogen, or C1-C4 alkyl, and/or
   R3 is selected from hydrogen, alkyl, cycloalkyl, or heterocycloalkyl, and/or
   R4 is selected from substituted or unsubstituted heteroaryl, or aryl;
whereby
   a cycloalkyl group denotes a non-aromatic ring system containing three to eight carbon atoms, wherein one or more of the carbon atoms in the ring can be substituted by a group being O, S, SO, SO₂, N or NR′;
   R′ is independently H, haloalkyl, hydroxyalkyl, alkyl, cycloalkyl, aryl, or heteroaryl;
   an aryl group denotes an aromatic group having five to ten carbon atoms, which can optionally be substituted by one or more substituents R″;
   R″ is independently H, —CO₂R′, —CONHR′, —CO-alkyl, —CN, alkyl, alkoxy, —OH, —SH, cycloalkyl, heterocycloalkyl, halogen, haloalkyl, haloalkyloxy, hydroxyalkyl, heteroaryl, or aryl
   a heteroaryl group denotes a five- or six-membered heterocyclic group which contains at least one heteroatom selected from O, N, and S and may be fused to another aromatic ring.

In a specific embodiment, the present invention provides for the HPIs according to general Formula (I) or pharmaceutically acceptable derivatives thereof, or a stereoisomer thereof as well as the pharmaceutically acceptable salts, solvates, and hydrates thereof, for use in a method of treating AD in a subject that is having HSV-1 infection and is having AD or is having HSV-1 infection and is suspected of having AD:
wherein
   R1 is selected from hydrogen, or C1-C4 alkyl, and/or
   R2 is selected from hydrogen, or C1-C4 alkyl, and/or
   R3 is selected from hydrogen, alkyl, cycloalkyl, or heterocycloalkyl, and/or
   R4 is selected from substituted or unsubstituted heteroaryl, or aryl;
whereby
   a cycloalkyl group denotes a non-aromatic ring system containing three to eight carbon atoms, wherein one or more of the carbon atoms in the ring can be substituted by a group being O, S, SO, SO₂, N or NR′;
   R′ is independently H, haloalkyl, hydroxyalkyl, alkyl, cycloalkyl, aryl, or heteroaryl;
   an aryl group denotes an aromatic group having five to ten carbon atoms, which can optionally be substituted by one or more substituents R″;
   R″ is independently H, —CO₂R′, —CONHR′, —CO-alkyl, —CN, alkyl, alkoxy, —OH, —SH, cycloalkyl, heterocycloalkyl, halogen, haloalkyl, haloalkyloxy, hydroxyalkyl, heteroaryl, or aryl
   a heteroaryl group denotes a five- or six-membered heterocyclic group which contains at least one heteroatom selected from O, N, and S and may be fused to another aromatic ring.

In another preferred embodiment, the present invention relates to the use of the HPIs according to general Formula (I), or pharmaceutically acceptable derivatives thereof, or a stereoisomer thereof as well as to pharmaceutically acceptable salts, solvates, and hydrates thereof, in a method of treating AD:
wherein
   R1 is selected from hydrogen, and/or
   R2 is selected from hydrogen, and/or
   R3 is selected from hydrogen, alkyl, or cycloalkyl, and/or
   R4 is selected from substituted or unsubstituted heteroaryl,
whereby
   a cycloalkyl group denotes a non-aromatic ring system containing three to eight carbon atoms, wherein one or more of the carbon atoms in the ring can be substituted by a group being O, S, SO, SO₂, N or NR′;
   R′ is independently H, haloalkyl, hydroxyalkyl, alkyl, cycloalkyl, aryl, or heteroaryl;
   an aryl group denotes an aromatic group having five to ten carbon atoms, which can optionally be substituted by one or more substituents R″;
   R″ is independently H, —CO₂R′, —CONHR′, —CO-alkyl, —CN, alkyl, alkoxy, —OH, —SH, cycloalkyl, heterocycloalkyl, halogen, haloalkyl, haloalkyloxy, hydroxyalkyl, heteroaryl, or aryl
   a heteroaryl group denotes a five- or six-membered heterocyclic group which contains at least one heteroatom selected from O, N, and S and may be fused to another aromatic ring.

In a specific embodiment, the present invention provides for the HPIs according to general Formula (I) or pharmaceutically acceptable derivatives thereof, or a stereoisomer thereof as well as the pharmaceutically acceptable salts, solvates, and hydrates thereof, for use in a method of treating AD in a subject that is having HSV-1 infection and is having AD or is having HSV-1 infection and is suspected of having AD:
wherein
   R1 is selected from hydrogen, and/or
   R2 is selected from hydrogen, and/or
   R3 is selected from hydrogen, alkyl, or cycloalkyl, and/or
   R4 is selected from substituted or unsubstituted heteroaryl,
whereby
   a cycloalkyl group denotes a non-aromatic ring system containing three to eight carbon atoms, wherein one or more of the carbon atoms in the ring can be substituted by a group being O, S, SO, SO₂, N or NR′;
   R′ is independently H, haloalkyl, hydroxyalkyl, alkyl, cycloalkyl, aryl, or heteroaryl;
   an aryl group denotes an aromatic group having five to ten carbon atoms, which can optionally be substituted by one or more substituents R″;
   R″ is independently H, —CO₂R′, —CONHR′, —CO-alkyl, —CN, alkyl, alkoxy, —OH, —SH, cycloalkyl, heterocycloalkyl, halogen, haloalkyl, haloalkyloxy, hydroxyalkyl, heteroaryl, or aryl
   a heteroaryl group denotes a five- or six-membered heterocyclic group which contains at least one heteroatom selected from O, N, and S and may be fused to another aromatic ring.

In a very specific embodiment, the present invention provides for the compound N-[5-(aminosulfonyl)-4-methyl-1,3-thiazol-2-yl]-N-methyl-2-[4-(2-pyridinyl)phenyl]acetamid

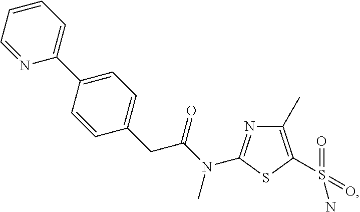 [SMILES: Cc1nc(N(C)C(=O)Cc2ccc(-c3ccccn3)cc2)sc1S(N)(=O)=O]

for use as HPI in a method of treating AD.

In another very specific embodiment, the present invention provides for the compound N-[5-(aminosulfonyl)-4-methyl-1,3-thiazol-2-yl]-N-methyl-2-[4-(2-pyridinyl)phenyl]acetamid

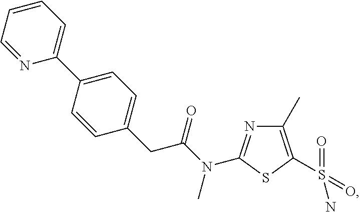 [SMILES: Cc1nc(N(C)C(=O)Cc2ccc(-c3ccccn3)cc2)sc1S(N)(=O)=O]

for use as HPIs in a method of treating AD in a subject that is having HSV-1 infection and is having AD or is having HSV-1 infection and is suspected of having AD.

In a further preferred embodiment of the present invention the HPIs according to general Formula (I) or pharmaceutically acceptable derivatives thereof, or a stereoisomer thereof as well as the pharmaceutically acceptable salts, solvates or hydrates thereof or functional equivalents thereof are intended for use in method of treating AD, in particular for use in method of treating AD in a subject that is having HSV-1 infection and is having AD or is having HSV-1 infection and is suspected of having AD, characterized in that AD is ameliorated by the inhibition of the HSV-1 helicase-primase complex.

The term “functional equivalent(s)” with the context of the HPIs according to general Formula (I) of the instant invention subsumes derivative compounds that are closely related to the explicit compounds according to general Formula (I), which however; exhibit the same biochemical function as HPIs, i.e. inhibiting the HSV-1 helicase-primase complex activity and thus function during HSV-1 DNA replication.

Surprisingly und unexpectedly, the herein provided HPIs exhibit superior efficacy in terms of inhibiting the accumulation of the key AD proteins Aβ and P-tau that occur during in vitro HSV-1 infection in cell culture assays, when directly compared to in vitro cell culture assays treated with the nucleoside analogue ACV.

In Vivo Imaging of Amyloid Plaques and/or Neurofibrillary Tangles/Therapy Monitoring

In vivo detection of pathological features of AD, namely the deposition of amyloid plaques/aggregates (containing Aβ) and the presence of neurofibrillary tangles (mainly composed of P-tau) in the brain of a subject, opens possibilities for detecting, differentiating and monitoring AD and monitoring the efficacy of methods of treatment of AD in said subject (Small G W. et al., In vivo brain imaging of tangle burden in humans. J Mol Neurosci. 19(3):323-7 (2002)).

In this regard, for instance a highly lipophilic tracer [¹⁸F]FDDNP was found to bind both, neurofibrillary tangles (containing P-tau) and the so-called senile plaques (containing Aβ) (Shoghi-Jadid K. et al, Localization of neurofibrillary tangles and beta-amyloid plaques in the brains of living patients with Alzheimer disease. Am J Geriatr Psychiatry 10:24-35, (2002)).

Moreover, while using positron-emission tomography (PET), it was reported that said tracer specifically labelled deposits of plaques and tangles in nine AD patients and seven comparison subjects (Nordberg A. PET imaging of amyloid in Alzheimer's disease. Lancet Neurol. 3:519-27, (2004)).

Thus, with the context of the present invention non-invasive methods for imaging and quantifying amyloid deposits (containing Aβ) and/or neurofibrillary tangles (containing P-tau) in vivo, enable the physician/clinician to monitor development or progress of AD, and to monitor the efficacy of the herein provided HPIs for use in methods of treatment of AD pursuant to the invention.

In accordance with the invention, potential ligands intended for visualizing amyloid plaques and/or neurofibrillary tangles in the brain of a subject have to show
i) a high binding affinity to amyloid-beta (Aβ) and/or P-tau, respectively, and
ii) have to be capable of crossing the blood-brain barrier (BBB).

Hence, in another preferred embodiment of the present invention the HPIs according to Formula (I) or functional equivalents thereof as well as the pharmaceutically acceptable salts, solvates or hydrates of the HPIs according to Formula (I) are intended for use in method of treating AD, in particular for use in method of treating AD in a subject that is having HSV-1 infection and is having AD or is having HSV-1 infection and is suspected of having AD,
characterized in that the accumulation of the characteristic senile plaques (containing Aβ) and/or neurofibrillary tangles (containing P-tau) in brain of said subject is stopped or reduced, when monitored by a clinical imaging technique selected from the group comprising magnetic resonance imaging (MRI), or positron emission tomography (PET-CT), or MRI/PET-CT imaging techniques, or single-photon emission computed tomography (SPECT),
after administration of an active pharmaceutical ingredient (API) according to general Formula (I) for at least 2 weeks, preferably 4 weeks, more preferably 6 weeks, even more preferably for at least 2 month after diagnosis of AD. This requires a comparable initial MRI, PET-CT, or both MRI/PET-CT, or SPECT imaging session at a time point (t0) prior to first administration of an API according to Formula (I).

Throughout the present specification and the claims, the expression “active pharmaceutical ingredient” and the corresponding abbreviation “API” with the context of the present invention denote a substance according to Formula (I) or functional equivalents thereof as well as the pharmaceutically acceptable salts, solvates or hydrates or derivatives and stereoisomers thereof in a pharmaceutical drug that is biologically active. In this context, “biologically active” means that said substance is a HPI according to the invention and thus inhibits the HSV-1 helicase-primase complex activity during HSV-1 DNA replication.

In another embodiment of the present invention the HPIs according to Formula (I) or functional equivalents thereof as well as the pharmaceutically acceptable salts, solvates or hydrates of HPIs according to Formula (I) are intended for use in method of treating AD, in particular for use in method of treating AD in a subject that is having HSV-1 infection and is having AD or is having HSV-1 infection and is suspected of having AD,
characterized in that the accumulation of the characteristic senile plaques (containing Aβ) and/or neurofibrillary tangles (containing P-tau) in brain of said subject is stopped or reduced, when determined by flow cytometry analysis for the detection and assessment of Aβ-oligomers and/or P-tau oligomers in cerebrospinal fluid from said subject,
after administration of an active pharmaceutical ingredient (API) according to Formula (I) for at least 2 weeks, preferably 4 weeks, more preferably 6 weeks, even more preferably for at least 2 month after diagnosis of AD. This requires a comparable initial flow cytometric analysis session at a certain time point (t0) prior to first administration of an API according to Formula (I).

It is further known that certain patients already suffering from MCI only gradually develop the full clinical symptoms of AD as outlined above. Thus, the invention also relates to methods as outlined above for the preventive treatment of patients having HSV-1 infection and suffering from MCI, who are thus in the prodromal state to may develop the full symptoms of AD.

In another preferred embodiment of the present invention pharmaceutical compositions are provided comprising at least one of the HPIs according to Formula (I) or functional equivalents thereof, or at least one pharmaceutically acceptable salt, solvate or hydrate of a HPI according to Formula (I) and at least one pharmaceutically acceptable carrier, excipient, solvent and/or diluent for use in method of treating the neurodegenerative disease AD, in particular for use in method of treating AD in a subject that is having HSV-1 infection and is having AD or is having HSV-1 infection and is suspected of having AD.

Suitable salts within the scope of the present invention for the HPIs of the Formula (I)—depending on substitution properties—are all acid addition salts or all salts with bases. Particular mention may be made of the pharmacologically tolerable inorganic and organic acids and bases customarily used in pharmacy.

Those suitable are, on the one hand, water insoluble and, particularly, watersoluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid such as (−)-L-malic acid or (+)-D-malic acid, fumaric acid, succinic acid, oxalic acid, tartaric acid such as (+)-L-tartaric acid or (−)-D-tartaric acid or meso-tartaric acid, embonic acid, stearic acid, toluenesulphonic acid, methanesulphonic acid or 3-hydroxy-2-naphthoicacid, the acids being employed in salt preparation—depending on whether a mono- or polybasic acid is concerned and depending on which salt is desired—in an equimolar quantitative ratio or one differing therefrom. Further, glutamate and aspartate are suitable salts of the instant invention.

Pharmacologically intolerable salts, which can be obtained, for example, as process products or by-products during the preparation of the compounds according to the present invention on an industrial scale, are converted into pharmacologically tolerable salts by processes known to the person skilled in the art.

According to expert's knowledge the HPIs of Formula (I) or functional equivalents thereof of the present invention as well as pharmaceutically acceptable salts of HPIs according to Formula (I) may contain, e.g. when isolated in crystalline form, varying amounts of solvents. Included within the scope of the invention are therefore all pharmaceutically acceptable solvates and in particular all pharmaceutically acceptable hydrates of the HPIs of Formula (I) as well as all solvates and in particular all hydrates of the pharmaceutically acceptable salts of the HPIs of Formula (I) or functional equivalents thereof.

In another preferred embodiment of the invention pharmaceutical formulations are provided comprising at least one API according to the Formula (I) or functional equivalents thereof, or a pharmaceutical acceptable salt, solvate or hydrate of a compound according to Formula (I), preferably in combination with one or more pharmaceutical acceptable excipients or carriers or diluents for use in a method of treating AD, in particular for use in method of treating AD in a subject that is having HSV-1 infection and is having AD or is having HSV-1 infection and is suspected of having AD.

Pharmaceutical acceptable carrier, excipients and/or diluents of the invention can be common inert carriers such as lactose, starch, sucrose, cellulose, magnesium stearate, dicalcium phosphate, calcium sulfate, talc, mannitol, ethyl alcohol (liquid filled capsules), suitable binders include starch, gelatin, natural sugars, corn sweeteners, natural and synthetic gums such as acacia, sodium alginate, carboxymethylcellulose, polyethylene glycol and waxes, sugars such as sucrose, starches derived from wheat corn rice and potato, natural gums such as acacia, gelatin and tragacanth, derivatives of seaweed such as alginic acid, sodium alginate and ammonium calcium alginate, cellulose materials such as methylcellulose, sodium carboxymethylcellulose and hydroxypropylmethyl-cellulose, polyvinylpyrrolidone, and inorganic compounds such as magnesium aluminum silicate; lubricants such as boric acid, sodium benzoate, sodium acetate, sodium chloride, magnesium stearate, calcium stearate, or potassium stearate, stearic acid, high melting point waxes, and other water soluble lubricants such as sodium chloride, sodium benzoate, sodium acetate, sodium oleate, polyethylene glycols and D, L-leucine; disintegrating agents (disintegrates) such as starch, methylcellulose, guar gum, modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses, and cross-linked micro-crystalline celluloses such as sodium croscaramellose, alginates such as alginic acid and sodium alginate, clays such as bentonites, and effervescent mixtures; coloring agents, sweetening agents, flavoring agents, preservatives; glidents are for example silicon dioxide and talc; suitable adsorbent are clay, aluminum oxide, suitable diluents are water or water/propylene glycol solutions for parenteral injections, juice, sugars such as lactose, sucrose, mannitol, and sorbitol, starches derived from wheat, corn rice, and potato, and celluloses such as microcrystalline cellulose.

The pharmaceutical compositions according to the invention preferably comprise 5 to 70%, more preferably 10 to 30% by weight of an API according to Formula (I) or functional equivalents thereof, or pharmaceutically acceptable salts, hydrates or solvates of a HPI according to Formula (I) (all percentage data are percentages by weight based on the weight of the pharmaceutical preparations). The pharmaceutical composition comprises usually 2 to 600 mg of an API according to Formula (I) or functional equivalents thereof, or pharmaceutically acceptable salts, hydrates or solvates of a compound according to Formula (I), preferably 5 to 500 mg, more preferably 10 to 300 mg and particularly preferably 20 to 200 mg based on a single daily dosage.

The compounds of the present invention for use in a method of treating AD, in particular for use in method of treating AD in a subject that is having HSV-1 infection and is having AD or is having HSV-1 infection and is suspected of having AD, can be applied systemically and/or locally. In another embodiment the pharmaceutical agents pursuant to the invention can be preferably applied orally.

For oral application tablets, capsules, dragées, granulate, pellets, powder, emulsions, suspensions, solutions and aerosols are preferred but not limited to.

Thus, in a preferred embodiment of the present invention HPIs according to Formula (I) or functional equivalents thereof, as well as pharmaceutically acceptable salts, solvates or hydrates of HPIs according to Formula (I), and pharmaceutical compositions thereof are provided for oral administration for use in method of treating AD, in particular for use in method of treating AD in a subject that is having HSV-1 infection and is having AD or is having HSV-1 infection and is suspected of having AD.

The compounds or compositions according to the invention can be administered to a patient in need thereof preferably once daily of about 100 mg of said compounds. The compounds or compositions according to the invention can also be administered to a patient in need thereof thrice daily, twice daily, once daily, thrice weekly, twice weekly, or once weekly.

Further particularly preferred embodiments of the invention are represented by the below consecutively numbered embodiments:
1) Helicase-primase inhibitor according to Formula (I)

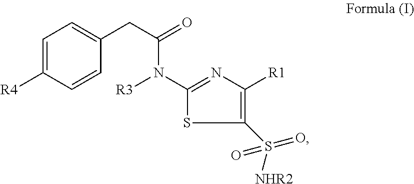

or pharmaceutically acceptable derivatives thereof, or a stereoisomer thereof, or pharmaceutically acceptable salts, solvates or hydrates thereof for use in a method of treating AD,
whereby
   R1 is selected from hydrogen, C1-C4 alkyl, or cycloalkyl, and/or
   R2 is selected from hydrogen, C1-C4 alkyl, or cycloalkyl, and/or
   R3 is selected from hydrogen, alkyl, cycloalkyl, heterocycloalkyl, haloalkyl, hydroxyalkyl, or alkoxyalkyl and/or,
   R4 is selected from substituted or unsubstituted heteroaryl, or aryl;
wherein
   a cycloalkyl group denotes a non-aromatic ring system containing three to eight carbon atoms, wherein one or more of the carbon atoms in the ring can be substituted by a group being O, S, SO, SO₂, N or NR′;
   R′ is independently H, haloalkyl, hydroxyalkyl, alkyl, cycloalkyl, aryl, or heteroaryl;
   an aryl group denotes an aromatic group having five to ten carbon atoms, which can optionally be substituted by one or more substituents R″;
   R″ is independently H, —CO₂R′, —CONHR′, —CO-alkyl, —CN, alkyl, alkoxy, —OH, —SH, cycloalkyl, heterocycloalkyl, halogen, haloalkyl, haloalkyloxy, hydroxyalkyl, heteroaryl, or aryl
   a heteroaryl group denotes a five- or six-membered heterocyclic group which contains at least one heteroatom selected from O, N, and S and may be fused to another aromatic ring.
2) Helicase-primase inhibitor according to embodiment 1), for use in a method of treating AD in a subject that is having HSV-1 infection and is having AD or is having HSV-1 infection and is suspected of having AD.
3) Helicase-primase inhibitor according to embodiment 2), wherein said subject is having HSV-1 infection and is suspected of having AD, when said subject shows at least the below manifested symptoms of mild cognitive impairment during clinical examination, i.e.
   a change in cognition
   impairment in one or more cognitive domains
   preservation of independence in functional abilities
   not demented,
and wherein said subject is positive for HSV-1 infection when clinically examined by HSV-test.
4) Helicase-primase inhibitor according to any of the embodiments 2) to 3), characterized in that said subject is positive for HSV-1 infection in an ex vivo HSV-test and possesses a specific genetic factor type 4 allele of the apolipoprotein E gene, i.e. APOE4 when said subject is positive for APOE4 in an ex vivo venous blood sample examined by APOE genotyping test.
5) Helicase-primase inhibitor according to any of the embodiments 2) to 3), characterized in that said subject is positive for HSV-1 infection in an ex vivo HSV-test and said subject is positive for PSEN1 in an ex vivo PSEN1 test.
6) Helicase-primase inhibitor according to any of the embodiments 2) to 3), characterized in that said subject is positive for HSV-1 infection in an ex vivo HSV-test and said subject is positive for the presence of Aβ42 and P-tau in an ex vivo Tau/Aβ42 test.
7) Helicase-primase inhibitor according to any of the embodiments 2) to 6), for use in a method of treating AD in a subject that is having HSV-1 infection and is having AD or is having HSV-1 infection and is suspected of having AD,
   whereby
      R1 is selected from hydrogen, or C1-C4 alkyl,
      R2 is selected from hydrogen, or C1-C4 alkyl,
      R3 is selected from hydrogen, alkyl, cycloalkyl, or heterocycloalkyl,
      R4 is selected from substituted or unsubstituted heteroaryl, or aryl.
8) Helicase-primase inhibitor according to any of the embodiments 2) to 7), for use in a method of treating AD in a subject that is having HSV-1 infection and is having AD or is having HSV-1 infection and is suspected of having AD,
   whereby
      R1 is selected from hydrogen,
      R2 is selected from hydrogen,
      R3 is selected from hydrogen, alkyl, or cycloalkyl,
      R4 is selected from substituted or unsubstituted heteroaryl.
9) Helicase-primase inhibitor N-[5-(aminosulfonyl)-4-methyl-1,3-thiazol-2-yl]-N-methyl-2-[4-(2-pyridinyl)phenyl]acetamid

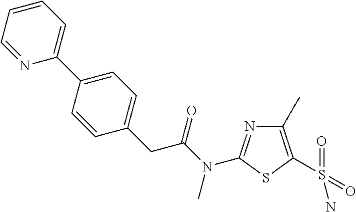 [SMILES: Cc1nc(N(C)C(=O)Cc2ccc(-c3ccccn3)cc2)sc1S(N)(=O)=O]

according to any of the embodiments 2) to 8), for use in a method of treating AD in a subject that is having HSV-1 infection and is having AD or is having HSV-1 infection and is suspected of having AD.
10) A pharmaceutical composition comprising at least one helicase-primase inhibitor according to any of the embodiments 2) to 9) and at least one pharmaceutically acceptable carrier, excipient, solvent and/or diluent for use in a method of treating AD in a subject that is having HSV-1 infection and is having AD or is having HSV-1 infection and is suspected of having AD.
11) Helicase-primase inhibitor according to any of the embodiments 1) to 9), or a composition according to embodiment 10) for oral administration.
12) Helicase-primase inhibitor according to any of the embodiments 2) to 8), for use in a method of treating AD in a subject that is having HSV-1 infection and is having AD or is having HSV-1 infection and is suspected of having AD, whereby said helicase-primase inhibitor is selected from crystalline N-[5-(aminosulfonyl)-4-methyl-1,3-thiazol-2-yl]-N-methyl-2-[4-(2-pyridinyl)-phenyl]acetamide mono methanesulfonic acid monohydrate particles of the following formula

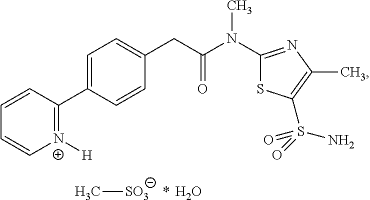

wherein said particles have a particle size range from 1 to 500 μm, a particle size distribution which is defined by d(0.1) from 2 to 100 μm, d(0.5) from 30 to 210 μm and d(0.9) from 70 to 400 μm and a specific surface area of less than 1.0 m²/g.
13) Helicase-primase inhibitor according to any of the embodiments 2) to 8) and 12), for use in a method of treating AD in a subject that is having HSV-1 infection and is having AD or is having HSV-1 infection and is suspected of having AD, wherein the N-[5-(aminosulfonyl)-4-methyl-1,3-thiazol-2-yl]-N-methyl-2-[4-(2-pyridinyl)-phenyl]acetamide mono methanesulfonic acid monohydrate particles of embodiment 12) have a particle size range from 2 μm to 400 μm.
14) Helicase-primase inhibitor according to any of the embodiments 2) to 8) and 12) to 13), for use in a method of treating AD in a subject that is having HSV-1 infection and is having AD or is having HSV-1 infection and is suspected of having AD, wherein the particles of any of the embodiments 12) to 13) have a particle size distribution which is defined by d(0.1) from 10 to 75 μm, d(0.5) from 100 to 175 μm, d(0.9) from 200 to 350 μm.
15) Helicase-primase inhibitor according to any of the embodiments 2) to 8) and 12) to 14), for use in a method of treating AD in a subject that is having HSV-1 infection and is having AD or is having HSV-1 infection and is suspected of having AD, wherein the particles of any of the embodiments 12) to 14) have a specific surface area of less than 0.3 m²/g.
16) A pharmaceutical composition comprising crystalline N-[5-(aminosulfonyl)-4-methyl-1,3-thiazol-2-yl]-N-methyl-2-[4-(2-pyridinyl)phenyl]acetamide mono methanesulfonic acid monohydrate particles as defined in any of the embodiments 12) to 15) and at least one pharmaceutically acceptable carrier, excipient, solvent and/or diluent.
17) The pharmaceutical composition according to embodiment 16), wherein the crystalline N-[5-(aminosulfonyl)-4-methyl-1,3-thiazol-2-yl]-N-methyl-2-[4-(2-pyridinyl)-phenyl]acetamide mono methanesulfonic acid monohydrate particles have a particle size range as defined in embodiment 13).
18) The pharmaceutical composition according to embodiment 16) or 17), wherein the crystalline N-[5-(aminosulfonyl)-4-methyl-1,3-thiazol-2-yl]-N-methyl-2-[4-(2-pyridinyl)-phenyl]acetamide mono methanesulfonic acid monohydrate particles have a particle size distribution as defined in embodiment 14).
19) The pharmaceutical composition according to any one of the embodiments 16) to 18), wherein the crystalline N-[5-(aminosulfonyl)-4-methyl-1,3-thiazol-2-yl]-N-methyl-2-[4-(2-pyridinyl)-phenyl]acetamide mono methanesulfonic acid monohydrate particles have a specific surface area as defined in embodiment 15).
20) The pharmaceutical composition according to any of the embodiments 16) to 19), for use in a method of treating AD in a subject that is having HSV-1 infection and is having AD or is having HSV-1 infection and is suspected of having AD, characterized by an absolute bioavailability of 70%±30% of the free base of N-[5-(aminosulfonyl)-4-methyl-1,3-thiazol-2-yl]-N-methyl-2-[4-(2-pyridinyl)-phenyl]acetamide, when administered in said composition containing at least 25 mg as free base equivalent of the crystalline N-[5-(aminosulfonyl)-4-methyl-1,3-thiazol-2-yl]-N-methyl-2-[4-(2-pyridinyl)-phenyl]acetamide mono methanesulfonic acid monohydrate to said subject that is having HSV-1 infection and is having AD or is having HSV-1 infection and is suspected of having AD.
21) The pharmaceutical composition according to any of the embodiments 16) to 20), for use in a method of treating AD in a subject that is having HSV-1 infection and is having AD or is having HSV-1 infection and is suspected of having AD, characterized by a mean maximum blood plasma concentration (mean Cₘₐₓ) of the free base of N-[5-(aminosulfonyl)-4-methyl-1,3-thiazol-2-yl]-N-methyl-2-[4-(2-pyridinyl)-phenyl]acetamide in said subject of at least one of
   a) 608±184 ng/ml for a 40 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
   b) 1306±125 ng/ml for a 80 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
   c) 2613±1341 ng/ml for a 160 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
   d) 3600±752 ng/ml for a 240 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
   e) 4648±1813 ng/ml for a 320 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
   f) 6926±1656 ng/ml for a 400 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
   g) 6921±2190 ng/ml for a 480 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered,
in said composition to said subject that is having HSV-1 infection and is having AD or is having HSV-1 infection and is suspected of having AD.
22) The pharmaceutical composition according to any of the embodiments 16) to 21), for use in a method of treating AD in a subject that is having HSV-1 infection and is having AD or is having HSV-1 infection and is suspected of having AD, characterized by a mean maximum blood plasma concentration (mean Cₘₐₓ) of the free base of N-[5-(aminosulfonyl)-4-methyl-1,3-thiazol-2-yl]-N-methyl-2-[4-(2-pyridinyl)-phenyl]acetamide in a subject of at least one of
   a) 608±184 ng/ml for a 40 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or characterized by an AUC₀₋₂₄ₕ of 10090±3114 ng-h/ml in a subject for a 40 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 72±3 h on average; said dosage being a single oral dose administered;

b) 1306±125 ng/ml for a 80 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or characterized by an AUC₀₋₂₄ₕ of 21940±2057 ng-h/ml in a subject for a 80 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 74±5 h on average; said dosage being a single oral dose administered;
c) 2613±1341 ng/ml for a 160 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or characterized by an AUC₀₋₂₄ₕ of 40470±16700 ng-h/ml in a subject for a 160 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 63±6 h on average; said dosage being a single oral dose administered;
d) 3600±752 ng/ml for a 240 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or characterized by an AUC₀₋₂₄ₕ of 59610±12770 ng-h/ml in a subject for a 240 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 64±5 h on average; said dosage being a single oral dose administered;
e) 4648±1813 ng/ml for a 320 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or characterized by an AUC₀₋₂₄ₕ of 76250±27630 ng-h/ml in a subject for a 320 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 57±3 h on average; said dosage being a single oral dose administered;
f) 6926±1656 ng/ml for a 400 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or characterized by an AUC₀₋₂₄ₕ of 104800±25740 ng-h/ml in a subject for a 400 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 57±4 h on average; said dosage being a single oral dose administered;
g) 6921±2190 ng/ml for a 480 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or characterized by an AUC₀₋₂₄ₕ of 112800±34260 ng-h/ml in a subject for a 480 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 53±4 h on average; said dosage being a single oral dose administered,
in said composition to said subject that is having HSV-1 infection and is having AD or is having HSV-1 infection and is suspected of having AD.
23) The pharmaceutical composition according to any of the embodiments 16) to 20), for use in a method of treating AD in a subject that is having HSV-1 infection and is having AD or is having HSV-1 infection and is suspected of having AD, characterized by a mean maximum blood plasma concentration at steady state (mean Cmax,ss) of the free base of N-[5-(aminosulfonyl)-4-methyl-1,3-thiazol-2-yl]-N-methyl-2-[4-(2-pyridinyl)-phenyl]acetamide in a subject of at least one of
   a) 1358±167 ng/ml for a 25 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a steady state dose after once daily single doses administered for 21 days;
   b) 6358±1701 ng/ml for a 100 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a steady state dose after once daily single doses administered for 21 days;
   c) 9987±2608 ng/ml for a 200 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a steady state dose after once daily single doses administered for 21 days,
in said composition to said subject that is having HSV-1 infection and is having AD or is having HSV-1 infection and is suspected of having AD.
24) The pharmaceutical composition according to any of the embodiments 16) to 20) and 23), for use in a method of treating AD in a subject that is having HSV-1 infection and is having AD or is having HSV-1 infection and is suspected of having AD, characterized by a mean maximum blood plasma concentration at steady state (mean C_{max,ss}) of the free base of N-[5-(aminosulfonyl)-4-methyl-1,3-thiazol-2-yl]-N-methyl-2-[4-(2-pyridinyl)-phenyl]acetamide in a subject of at least one of
   a) 1358±167 ng/ml for a 25 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or characterized by an AUC_{τ,ss} of 23430±3020 ng-h/ml in a subject for a 25 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 69±6 h on average, said dosage being a steady state dose after once daily single doses administered for 21 days;
   b) 6358±1701 ng/ml for a 100 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or characterized by an AUC_{τ,ss} of 108800±28610 ng-h/ml in a subject for a 100 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 60±4 h on average, said dosage being a steady state dose after once daily single doses administered for 21 days,
in said composition to said subject that is having HSV-1 infection and is having AD or is having HSV-1 infection and is suspected of having AD.
25) The pharmaceutical composition of embodiment 20), wherein said absolute bioavailability is achieved in a human.
26) The pharmaceutical composition of the embodiments 16) to 24), wherein said mean Cₘₐₓ and C_{max,ss} is achieved in a human.
27) The pharmaceutical composition of the embodiments 22) and 24), wherein said AUC₀₋₂₄ₕ and t_{1/2z} is achieved in a human.
28) The pharmaceutical composition of the embodiment 24), wherein said AUC_{τ,ss} and t_{1/2z} is achieved in a human.

### DEFINITIONS

Throughout the specification and the claims, generally designated as “Alzheimer's disease” or “AD” are conditions, which have—as a common feature—the loss of acquired intellectual capacities in a subject, above all regarding memory and normal personality level, as a result of damage to the brain. In accordance with the invention patients suffering from MCI are susceptible to convert to AD, and thus are fully comprised by the scope of the invention as patient group.

The term “pharmaceutical acceptable” in the context of the invention means that the relevant derivatives, functional equivalents, salts, solvates, hydrates, excipients, carrier, diluents, and solvents according to the invention are safe and effective for the comprised use in mammals and that possess the desired biological activity and/or function, respectively.
i) In general, the terms “functional derivative(s)” and/or “functional equivalent(s)” refer to a compound or compound(s) related to the HPIs according to the general Formula (I) that differ/s at least in one atom when compared to the HPIs of Formula (I); however, that exhibit/s the same inhibitory efficacy for the accumulation of Aβ and P-tau, i.e. inhibiting the HSV-1 helicase-primase complex activity during HSV-1 DNA replication.

The “cerebrospinal fluid” is a clear colorless bodily fluid produced in the choroid plexus of the brain. It acts as a cushion or buffer for the cortex, providing a basic mechanical and immunological protection to the brain inside the skull and serves a vital function in cerebral autoregulation of cerebral blood flow. It should be emphasized that in accordance with the invention ex vivo samples of cerebrospinal fluid have to be taken for conducting flow cytometry analysis for monitoring Aβ-oligomers and/or P-tau oligomers.

The term “flow cytometry analysis” denotes a laser based, biophysical technology used e.g. for cell counting, cell sorting, biomarker detection and for protein engineering via suspension of cells in a fluid stream, and passing said stream by an electronic detection apparatus. It allows simultaneous multiparametric analysis of the physical and/or chemical characteristics of up to thousands of particles per second.

In accordance with the invention flow cytometry analysis is intended for diagnosis of AD and/or monitoring of the development or progress of AD through physical assorting of Aβ-oligomers and/or P-tau oligomers based on their physical and/or chemical properties in ex vivo samples of cerebrospinal fluid of a subject that is having HSV-1 infection and is suspected of having AD.

The “active pharmaceutical ingredient” or “API” denotes the substance(s) or compound(s) according to the general Formula (I) or pharmaceutical derivative(s), stereoisomer(s) or functional equivalent(s) thereof, as well as pharmaceutically acceptable salts, solvates or hydrates of compounds according to general Formula (I) in a pharmaceutical drug pursuant to the invention that is biologically active and thus HPIs for the HSV-1 helicase-primase complex.

The abbreviation “MRI” denotes a diagnostic or monitoring tool also referred to as magnetic resonance imaging, nuclear magnetic resonance imaging (NMRI), or magnetic resonance tomography (MRT) for the purpose of medical imaging used in radiology to visualize internal structures of the body in detail. MRI makes use of the property of nuclear magnetic resonance (NMR) to image nuclei of atoms inside the body. Specifically, MRI provides good contrast between the different soft tissues of the body, which is especially useful in imaging the brain and associated structures. MRI does not use ionizing radiation.

The abbreviation “PET-CT” denotes a nuclear medicine imaging technique, being positron emission computerized-tomography that produces a 3D image of functional processes within the body of a subject. A PET-CT apparatus/scanner detects pairs of gamma rays emitted indirectly by a positron-emitting radionuclide (commonly referred to as PET-tracer) that is introduced into the body in combination with a biologically active molecule. In accordance with the invention said biologically active molecule can be related to any useful molecule in detecting senile plaques (amyloid aggregates) in brain of a subject. Three-dimensional images of PET-tracer concentration in the body of a subject are then constructed by computer-assisted analysis. In specific scanners, 3D imaging is often accomplished with the aid of a CT X-ray scan performed on the patient during the same session, in the same apparatus.

The abbreviation “MRI/PET-CT” denotes a combination imaging technique based on simultaneous or consecutive performance of an MRI and PET-CT scan, which results in images that provide for morphological and functional analysis in both.

The abbreviation “SPECT” denotes a nuclear medicine tomographic imaging method based on gamma rays. This technique is able to provide for real 3D information. The technique requires gamma-emitting radioisotope (called radionuclide) to be injected into the bloodstream of a patient. Hereby, the radioisotope is a soluble dissolved ion, which also has chemical properties that allow it to be concentrated in regions or pathways of medical interest for disease detection.

### FIGURE DESCRIPTION

FIG. 1—The exemplary compound according to Formula (I) is more effective than ACV at reducing the HSV-1 protein levels as well as accumulation of AD proteins Aβ and P-tau after HSV-1 infection in Vero cells

Vero cells were infected with HSV-1 at 0.01 plaque forming units (pfu) per cell [pfu/cell] for 24 hours and treated with varying concentrations of ACV and/or the exemplary compound according to Formula (I). Subsequently, cells were fixed and examined for HSV-1 proteins (A), Aβ(B) and P-tau (C) by immunocytochemistry. 1=vehicle; 2-5=increasing concentrations of the exemplary compound according to Formula (I) [0.025, 0.25, 2.5, 25 μM]; 6-9=increasing concentrations of acyclovir [0.025, 0.25, 2.5, 25 μM].

FIG. 2—The exemplary compound according to Formula (I) is more effective than ACV at reducing the HSV-1 protein levels as well as accumulation of AD proteins Aβ and P-tau after HSV-1 infection in SH-SY5Y cells

SH-SY5Y cells were infected with HSV-1 at 0.01 plaque forming units (pfu) per cell [pfu/cell] for 24 hours and treated with varying concentrations of ACV and/or the exemplary compound according to Formula (I). Subsequently, cells were fixed and examined for HSV-1 proteins (A), Aβ (B) and P-tau (C) by immunocytochemistry. 1=vehicle; 2-5=increasing concentrations of the exemplary compound according to Formula (I) [0.025, 0.25, 2.5, 25 μM]; 6-9=increasing concentrations of acyclovir [0.025, 0.25, 2.5, 25 μM].

FIG. 3 shows the relationship between single doses [mg] (5 mg-600 mg as free base equivalent) of tablets containing the specific crystalline N-[5-(aminosulfonyl)-4-methyl-1,3-thiazol-2-yl]-N-methyl-2-[4-(2-pyridinyl)phenyl]acetamide mono methanesulfonic acid monohydrate salt, wherein the particle size distribution is preferably defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g, and AUCinf [ng·h/mL](identical to AUC_{0-∞}), measured as the free base of N-[5-(aminosulfonyl)-4-methyl-1,3-thiazol-2-yl]-N-methyl-2-[4-(2-pyridinyl)phenyl]acetamide.

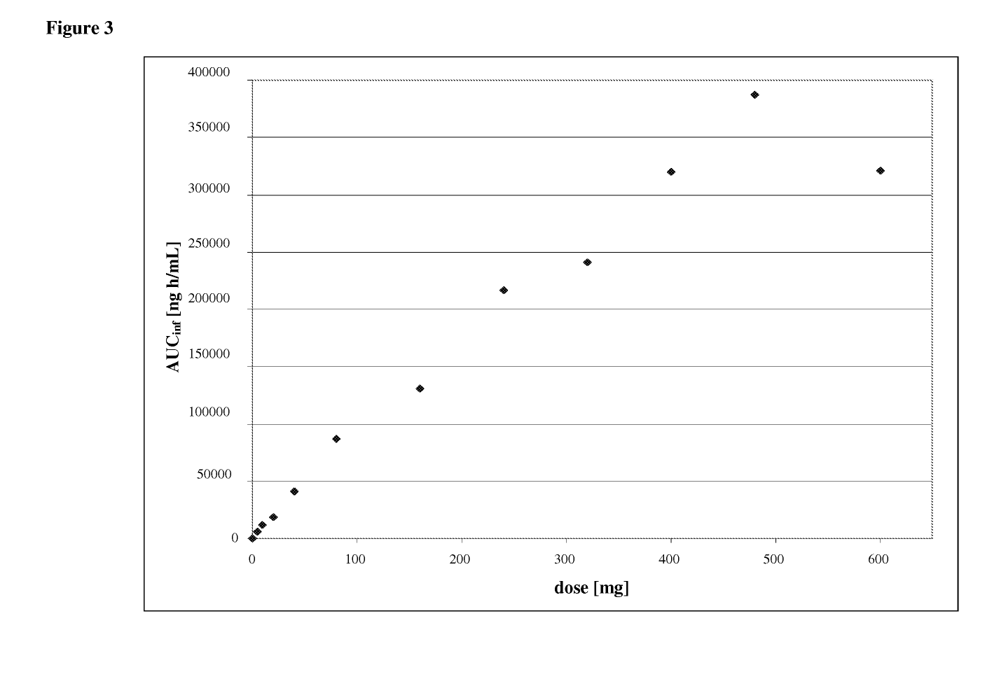

FIG. 4 shows plasma time curves of the specific free base of N-[5-(aminosulfonyl)-4-methyl-1,3-thiazol-2-yl]-N-methyl-2-[4-(2-pyridinyl)phenyl]acetamide measured by HPLC in plasma of healthy male volunteers (n=6) after a single oral dose of tablets containing 5 mg; 10 mg; 20 mg and 40 mg as free base equivalent of crystalline N-[5-(aminosulfonyl)-4-methyl-1,3-thiazol-2-yl]-N-methyl-2-[4-(2-pyridinyl)phenyl]acetamide mono methanesulfonic acid monohydrate, wherein the particle size distribution is preferably defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g. The four different doses were administered as immediate release tablets and blood was collected at indicated time points after administration. The free base concentration was measured by HPLC in plasma. The EC90 derived from cell culture was corrected for protein binding taking into account the fraction unbound of N-[5-(aminosulfonyl)-4-methyl-1,3-thiazol-2-yl]-N-methyl-2-[4-(2-yridinyl)phenyl]acetamide mono methanesulfonic acid monohydrate in cell culture medium (71%) and in murine plasma (2.8%). Plasma concentrations remained over the EC₉₀ for the entire treatment interval after administration of 40 mg as free base equivalent of crystalline N-[5-(aminosulfonyl)-4-methyl-1,3-thiazol-2-yl]-N-methyl-2-[4-(2-yridinyl)phenyl]acetamide mono methanesulfonic acid monohydrate once daily at steady state. EC90 denotes 90% effective concentration.

FIG. 5 shows plasma time curve of the free base of N-[5-(aminosulfonyl)-4-methyl-1,3-thiazol-2-yl]-N-methyl-2-[4-(2-pyridinyl)phenyl]acetamide after administration of tablets containing 5 mg and 25 mg as free base equivalent of crystalline N-[5-(aminosulfonyl)-4-methyl-1,3-thiazol-2-yl]-N-methyl-2-[4-(2-yridinyl)phenyl]acetamide mono methanesulfonic acid monohydrate, wherein the particle size distribution is preferably defined by d(0.1) from 2 to 100 μm, d(0.5) from 30 to 210 μm and d(0.9) from 70 to 400 μm with a specific surface area of the particles less than 1.0 m2/g, and more preferably defined by d(0.1) from 10 to 75 μm, d(0.5) from 100 to 175 μm, d(0.9) from 200 to 350 μm with a specific surface area of the particles less than 0.3 m²/g in fasted, male healthy volunteers (n=12) after multiple dose administration thereof once daily at day 21 (steady state). The two different doses were administered as immediate release tablets and blood was collected at indicated time points after administration. The free base concentration was measured by HPLC in plasma. The EC₉₀ derived from cell culture was corrected for protein binding taking into account the fraction unbound of N-[5-(aminosulfonyl)-4-methyl-1,3-thiazol-2-yl]-N-methyl-2-[4-(2-yridinyl)phenyl]acetamide mono methanesulfonic acid monohydrate in cell culture medium (71%) and in murine plasma (2.8%). Plasma concentrations remained over the EC₉₀ for the entire treatment interval after administration of 25 mg as free base equivalent of crystalline N-[5-(aminosulfonyl)-4-methyl-1,3-thiazol-2-yl]-N-methyl-2-[4-(2-yridinyl)phenyl]acetamide mono methanesulfonic acid monohydrate once daily at steady state. EC₉₀ denotes 90% effective concentration.

FIG. 6 shows dose proportionality after single dose administration to fasted male healthy volunteers (n=6) of up to 400/480 mg as free base equivalent of the specific crystalline N-[5-(aminosulfonyl)-4-methyl-1,3-thiazol-2-yl]-N-methyl-2-[4-(2-yridinyl)phenyl]acetamide mono methanesulfonic acid monohydrate salt, wherein the particle size distribution is preferably defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g. Terminal half-life (t_{1/2z}) is between 52 h and 85 h.

FIG. 7 shows that the total exposure of the free base of N-[5-(aminosulfonyl)-4-methyl-1,3-thiazol-2-yl]-N-methyl-2-[4-(2-yridinyl)phenyl]acetamide, resultant from crystalline mono methanesulfonic acid monohydrate administration with 80 mg single dose as free base equivalent, wherein the particle size distribution is preferably defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g to woman is higher (triangle) compared to males (dots) for n=6. After normalization to body weight no relevant gender differences could be revealed.

### EXAMPLES

The following helicase-primase complex inhibitory compound N-[5-(aminosulfonyl)-4-methyl-1,3-thiazol-2-yl]-N-methyl-2-[4-(2-pyridinyl)phenyl]acetamid is based on the Markush Formula (I), and was tested for its inhibitory efficacy pursuant to the invention upon induced HSV-1 infection in vitro. The inhibitory efficacy was evaluated for Aβ and P-tau staining in the tested cells:

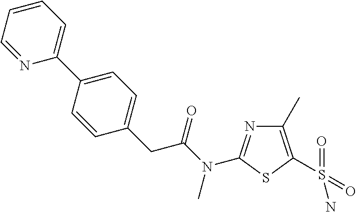 [SMILES: Cc1nc(N(C)C(=O)Cc2ccc(-c3ccccn3)cc2)sc1S(N)(=O)=O]

N-[5-(aminosulfonyl)-4-methyl-1,3-thiazol-2-yl]-N-methyl-2-[4-(2-pyridinyl)phenyl]acetamid (hereinafter referred to as exemplary compound of Formula (I)).

### Study Design

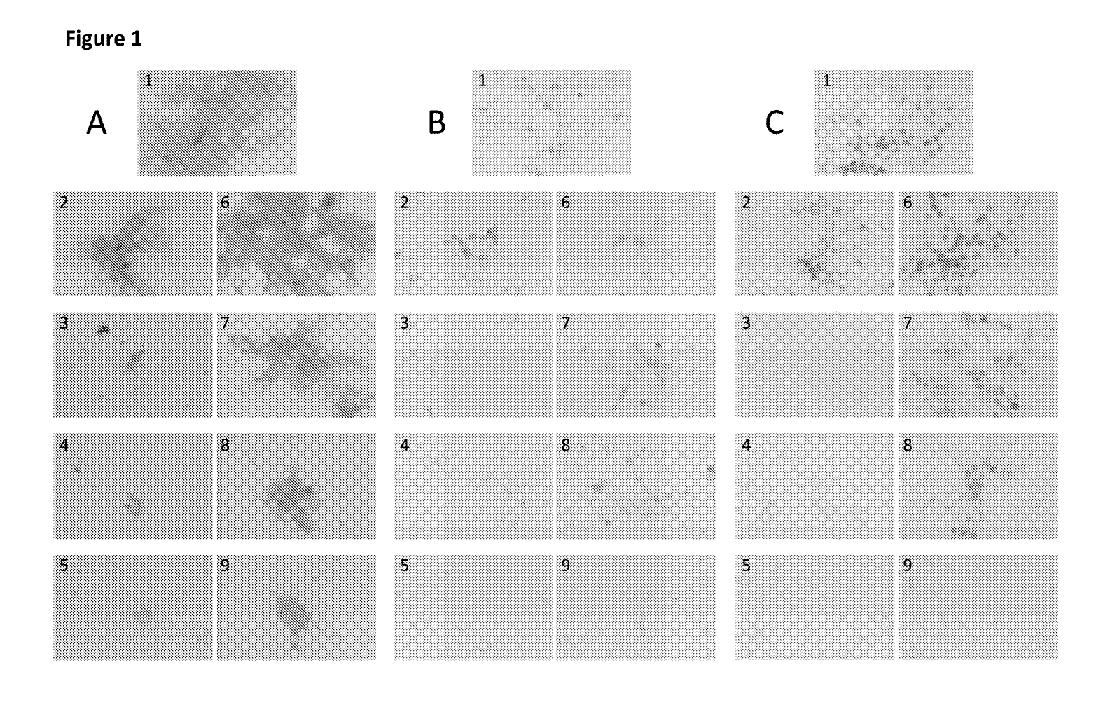

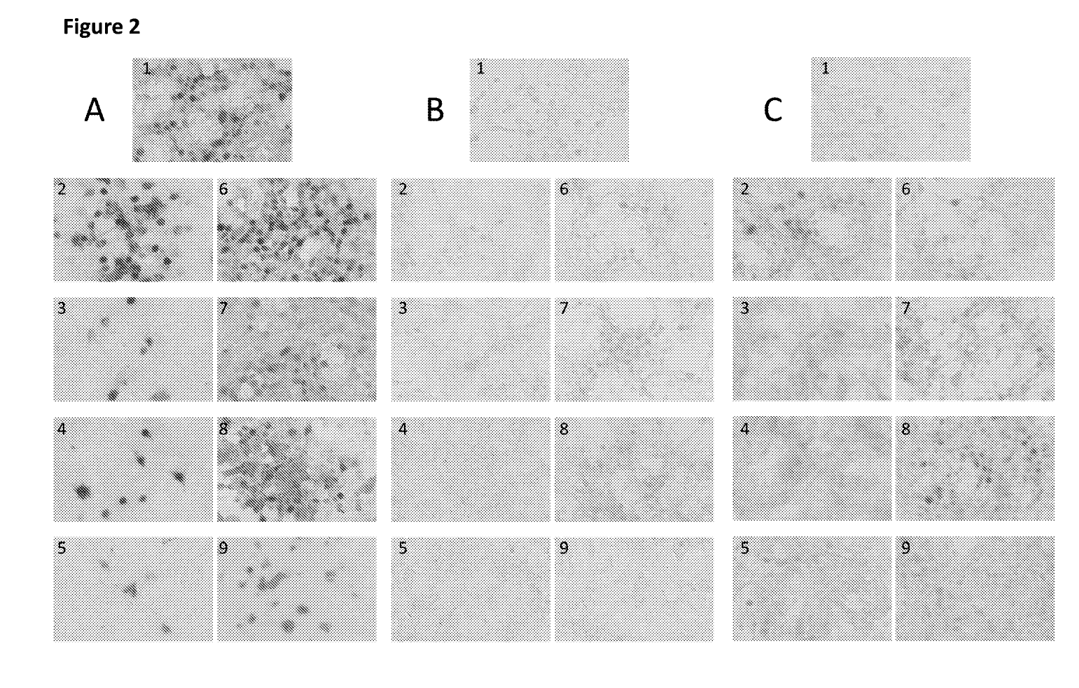

The inventors have compared the efficacy of the exemplary compound according to Formula (I) with that of ACV in respect to Aβ and P-tau accumulation in HSV-1 infected cells. African green monkey kidney (Vero) cells or human neuroblastoma (SH-SY5Y) cells were infected with HSV-1 at a multiplicity of infection (MOI) of 0.01 plaque forming units (pfu) per cell [pfu/cell] for 24 hours and were treated with the exemplary compound according to Formula (I), or ACV. The antiviral agents were added at the time of infection and remained throughout the infection. Infection led to typical cytopathic effects (clustering of infected cells positive for HSV-1 protein staining (FIGS. 1A and 2 A, panel 1)).

### Results

For both cell lines, treatment with the herein provided compound according to Formula (I) reduced the cluster size of HSV-1 infected cells, and the number of stained cells in clusters dose dependently: at 0.025 μM (FIGS. 1 A and 2 A, panel 2), clusters were slightly smaller compared to control (FIGS. 1 A and 2 A, panel 1) but with increasing concentrations of the herein provided compound according to Formula (I) clusters disappear and only occasionally infected cells could be detected (FIGS. 1 A and 2 A, panel 3-5). Consistently, staining for Aβ and for P-tau was reduced by the compound according to Formula (I) but surprisingly in a more prominent fashion compared to HSV-1 proteins (FIGS. 1 B and 2 B, panel 2-5 for Aβ and FIGS. 1 C and 2 C, panel 2-5 for P-tau). No staining was observed in mock-infected cells (data not shown). Treatment with equimolar concentrations of ACV also resulted in smaller clusters of infected cells but the effect was not as strong as with the exemplary compound according to Formula (I) (FIGS. 1 A and 2 A, panel 6-9): 0.025 μM ACV had little effect on cluster sizes, whereas higher ACV concentrations reduced their sizes but less so than did the treatment with equimolar exemplary compound according to Formula (I). The lower efficacy of ACV compared with the exemplary compound according to Formula (I) became even more obvious for Aβ and P-tau, as staining for these molecules was still visible at higher concentrations of ACV, i.e. the reduction was less effective than with the exemplary compound according to Formula (I) (FIGS. 1 B and 2 B, panel 6-9 for Aβ and FIGS. 1 C and 2 C, panel 6-9 for P-tau).

### Conclusion

The immunocytochemical results clearly show that the exemplary compound according to Formula (I) is notably more efficient than ACV in terms of reducing the amount of the key AD proteins, Aβ and P-tau.

Besides and also expectedly, the HSV-1 proteins in Vero cells (FIG. 1) and even more so in SH-SY5Y cells (FIG. 2) are reduced by the exemplary compound of Formula (I). The difference was marked at the experimental conditions (infection for 24 hours at a MOI of 0.01 pfu/cell), which to some extent might mirror the presumed low HSV level in human brains upon HSV-1 reactivation. A total or almost total removal of antibody staining to all these proteins/peptides could be achieved remarkably with the exemplary compound according to Formula (I). The reduction at the concentration used in the test with the exemplary compound according to Formula (I) was superior to ACV at the HSV-1 MOI of 0.01 pfu/cell.

In conclusion, the compounds according to Formula (I) or functional equivalents or pharmaceutically acceptable salts, hydrates or solvates thereof represent an alternative treatment option for HSV-1 with a different mode of action compared to ACV, and the compounds pursuant to the invention are remarkably more effective at combating the HSV-1 induced AD protein formation of Aβ and P-tau.

### Example a

TABLE a
Exemplary formulations of 25 mg and 100 mg dose strengths for the below
testings (calculated as free base form; i.e. the free base equivalent thereof)

| | mg per 25 mg | mg per 100 mg |
|---|---|---|
| Component | tablet | tablet |
| N-[5-(aminosulfonyl)-4-methyl- | 32.3 | 129.0 |
| 1,3-thiazol-2-yl]-N-methyl-2[4- | (salt) | (salt) |
| (2-pyridinyl)-phenyl]acetamide | | |
| mono methanesulfonic acid | | |
| monohydrate | | |
| Microcrystalline | 60.9 | 243.4 |
| cellulose | | |
| Croscarmellose | 9.8 | 39.0 |
| sodium | | |
| Mannitol | 20.0 | 80.1 |
| Silica, colloidal | 1.3 | 5.0 |
| anhydrous | | |
| Magnesium stearate | 0.9 | 3.5 |
| Sum final blend | 125.2 | 500.0 |

These exemplary formulations were adapted in dependence of the amount of N-[5-(aminosulfonyl)-4-methyl-1,3-thiazol-2-yl]-N-methyl-2-[4-(2-pyridinyl)-phenyl]acetamide mono methanesulfonic acid monohydrate applied during testing.

### Single-Dose Escalation and Pharmacokinetics

An advantage of the tablets containing the specific crystalline form of N-[5-(aminosulfonyl)-4-methyl-1,3-thiazol-2-yl]-N-methyl-2-[4-(2-pyridinyl)-phenyl]acetamide mono methanesulfonic acid monohydrate according to the invention is that these tablets will have an optimised dissolution rate based on the particle size distribution of the crystalline form of N-[5-(aminosulfonyl)-4-methyl-1,3-thiazol-2-yl]-N-methyl-2-[4-(2-pyridinyl)-phenyl]acetamide mono methanesulfonic acid monohydrate, wherein the particle size distribution is preferably defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g, and thus, the drug may be absorbed into the blood stream much faster compared to N-[5-(aminosulfonyl)-4-methyl-1,3-thiazol-2-yl]-N-methyl-2-[4-(2-pyridinyl)-phenyl]acetamide as crystalline free base form. Furthermore, the surprising dispersion times obtained with tablets according to the invention are advantageous for swallowable tablets. In a further embodiment, the tablets according to the invention can be presented for dispersion in water.

Therefore, pharmacokinetic studies in human subjects were undertaken following both single and multiple dose administrations of the crystalline mono mesylate monohydrate salt of N-[5-(aminosulfonyl)-4-methyl-1,3-thiazol-2-yl]-N-methyl-2-[4-(2-pyridinyl)phenyl]acetamide, wherein the particle size distribution is preferably defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g.

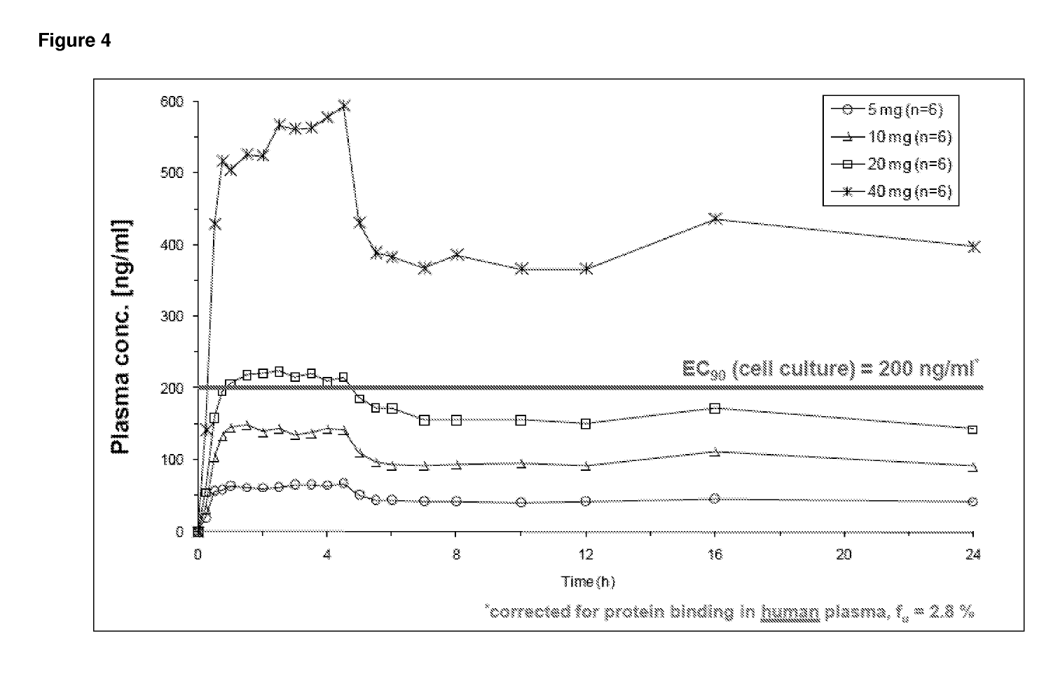

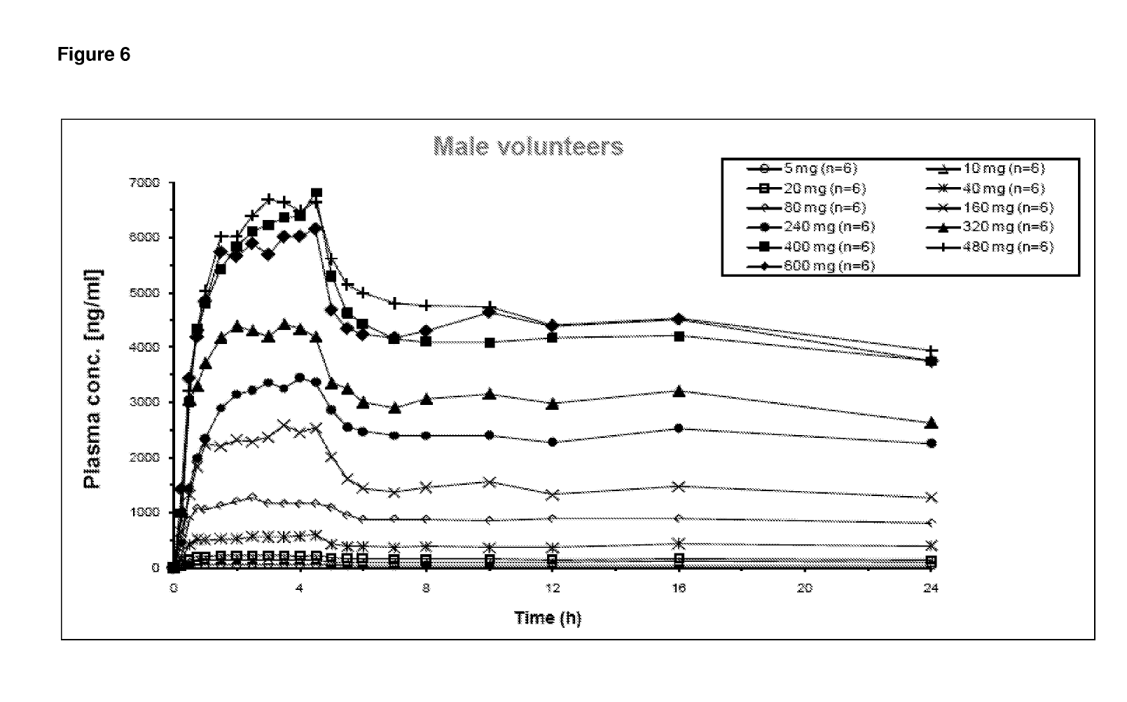

Single oral doses of the crystalline mono mesylate monohydrate salt of N-[5-(aminosulfonyl)-4methyl1,3thiazol-2-yl]-N-methyl-2-[4-(2-pyridinyl)phenyl]acetamide using the formulations in accordance with example a and Table a, were administered to six volunteers per dose step. The overall shape of the plasma concentrations vs. time profiles were similar across all doses applied (see FIG. 4 and FIG. 6).

There was a rapid and continuous increase of plasma concentrations of the free base of N-[5-(aminosulfonyl)4methyl1,3thiazol-2-yl]-N-methyl-2-[4-(2-pyridinyl)phenyl]acetamide switching into a period of markedly slower absorption rate and evidence of a plateau effect in exposure. Thereafter, for all doses investigated, there was a decrease of concentrations of the free base of N-[5-(aminosulfonyl)4methyl1,3thiazol-2-yl]-N-methyl-2-[4-(2-pyridinyl)phenyl]acetamide starting after 4.0 to 4.5 hours post administration of the crystalline mono mesylate monohydrate salt of N-[5-(aminosulfonyl)4methyl1,3thiazol-2-yl]-N-methyl-2-[4-(2-pyridinyl)phenyl]acetamide. This phase was followed by a phase of prolonged exposure, which was characterized by a long half-life, which is favorable for the treatment of infectious diseases. The mean terminal elimination half-life (t_{1/2z}) ranged between 52 h and 85 h.

For doses from 5 mg to 480 mg as free base equivalent there was a dose-proportional increase in AUC_{0-∞} (AUC, area under curve); a single dose of 600 mg as free base equivalent did not cause any further rise of exposure as shown by AUC_{0-∞} (see FIG. 4 and FIG. 6).

Maximum plasma concentrations were linearly related to doses from 5 mg to 400 mg as free base equivalent. At the higher dose up to 600 mg as free base equivalent no further increase of exposure was obtained as shown by the both Cₘₐₓ and AUC_{0-∞}. Median tₘₐₓ ranged from 1.5 to 4.25 hours without any obvious relation to dose. A summary of single-dose pharmacokinetic parameters is shown in Table b.

Table b: Pharmacokinetic parameters after ascending single oral doses of crystalline mono mesylate monohydrate salt of N-[5-(aminosulfonyl)-4-methyl-1,3-thiazol-2-yl]-N-methyl-2-[4-(2-pyridinyl)phenyl]acetamide in the formulations in accordance with table a wherein the particle size distribution is preferably defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g.

In the following the abbreviations used in Table b are defined.

AUC_{0-∞}: Area under the analyte vs. time concentration from time of administration up to infinity, calculated as

$${AUC}_{0 - ∞} = {AUC}_{0 - last} + \frac{{C}_{last}}{{λ}_{z}}$$

wherein AUC₀₋ₗₐₛₜ is defined as the area under the analyte vs. time concentration up to time of the last qualifiable concentration, calculated by linear up/log down summation and Cₗₐₛₜ is defined as last quantifiable observed analyte concentration. λ_{z} is the apparent terminal elimination rate constant, determined by linear regression of terminal points of In-linear analyte concentration-time curve.

Cₘₐₓ is the maximal observed analyte concentration and tₘₐₓ is the time to reach Cₘₐₓ;

t_{1/2z} is defined as the apparent terminal elimination half-life, calculated as

$${t}_{1 / 2 z} = \frac{ln ( 2 )}{{λ}_{z}} ,$$

wherein λ_{z} is defined as above.

MRT: Mean Residence Time; calculated AUMC divided by AUC, wherein AUMC is the area under the first moment of the concentration-time curve from zero up to ∞ with extrapolation of the terminal phase and AUC is the area under the concentration-time curve from zero up to ∞ with extrapolation of the terminal phase.

CL/F refers to the clearance after oral administration of a drug and Aₑ refers to the amount of drug excreted in the urine.

TABLE b

| | parameter (means; n = 6 volunteers/dose) | | | | | | |
|---|---|---|---|---|---|---|---|
| dose [mg] | AUC_{0-∞} | | | | | | Aₑ |
| free base | [ng · | Cₘₐₓ | tₘₐₓ^{a} | t_{1/2z} | MRT | CL/F | [% of |
| equivalent | h/mL] | [ng/mL] | [h] | [h] | [h] | [L/h] | dose] |
| 5 | 5800 | 74 | 4.00 | 80 | 117 | 0.89 | 0 |
| 10 | 11670 | 170 | 1.50 | 85 | 116 | 0.87 | 0 |
| 20 | 18540 | 234 | 2.77 | 76 | 105 | 1.10 | 0 |
| 40 | 40680 | 608 | 3.50 | 72 | 94 | 1.00 | 0 |
| 80, | 87220 | 1306 | 2.50 | 74 | 94 | 0.94 | 0.16 |
| males | (99790)^{b} | (1499)^{b} | | | | | |
| 80, | 96230 | 1999 | 4.00 | 58 | 77 | 0.85 | 0.31 |
| females | (90050)^{b} | (1853)^{b} | | | | | |
| 160 | 130800 | 2613 | 3.50 | 63 | 83 | 1.28 | 0.15 |
| 240 | 216900 | 3600 | 4.00 | 64 | 82 | 1.15 | 0.21 |
| 320 | 241100 | 4648 | 2.25 | 57 | 70 | 1.47 | 0.16 |
| 400 | 320300 | 6926 | 4.25 | 57 | 64 | 1.31 | 0.15 |
| 480 | 387200 | 6921 | 3.25 | 53 | 72 | 1.33 | 0.26 |
| 600 | 320800 | 6442 | 4.25 | 52 | 65 | 2.00 | 0.09 |

^{a}for tₘₐₓ the median is given,
^{b}value normalized to body weight for a 70 kg subject;

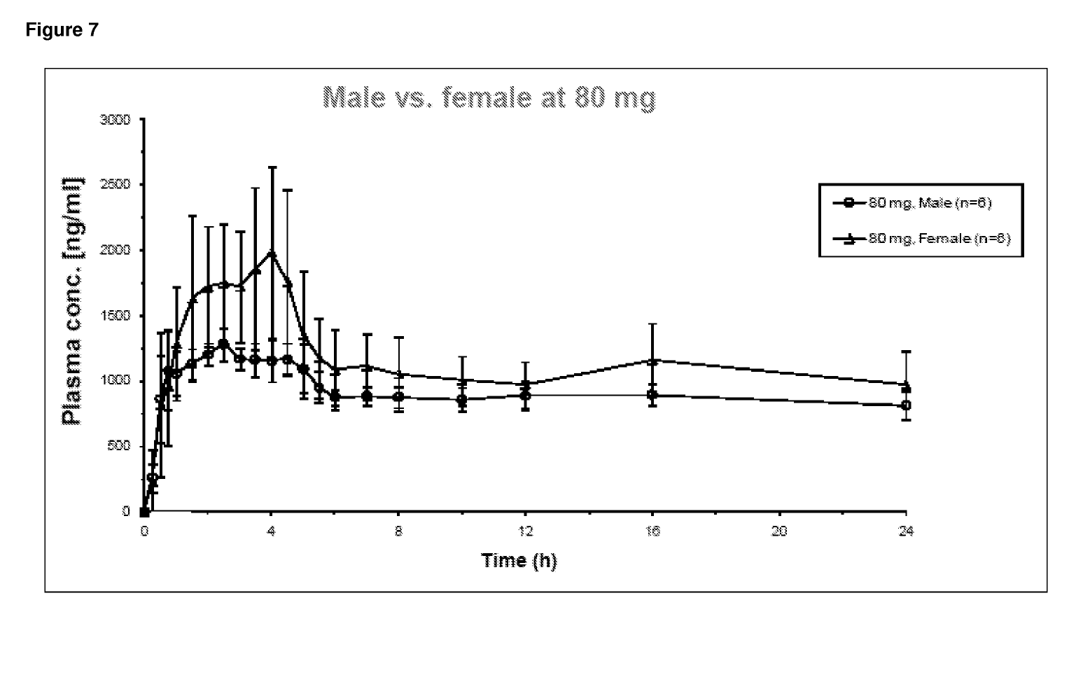

Based on data for the 80 mg dose as free base equivalent (see also FIG. 7), women appeared to exhibit a higher exposure compared to males according to AUC_{0-∞} and Cₘₐₓ (see Table b). However, normalization to body weight revealed that this apparent difference could be explained by the lower body weight of the female volunteers compared to males (see FIG. 7).

Summary of the Results of Example a:

For doses from 5 mg to 400 and 480 mg as free base equivalent, respectively, there was a linear, i.e. dose-proportional, increase in AUC_{0-∞} and Cₘₐₓ with dose; higher doses do not further increase the exposure. The mean terminal elimination half-life (t_{1/2z}) ranged between 52 h and 85 h. No clinical relevant gender-related difference in exposure was detected for a single dose of 80 mg as free base equivalent (see FIG. 7).

### Example b

### Multiple-Dose Escalation and Pharmacokinetics

For three doses of the mono mesylate monohydrate salt of N-[5-(aminosulfonyl)-4-methyl-1,3-thiazol-2-yl]-N-methyl-2-[4-(2-pyridinyl)phenyl]acetamide in formulations in accordance with table a, wherein the particle size distribution is preferably defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g, investigated (5, 25, and 100 mg as free base equivalent; once per day oral administration, 20 days), the individual concentration-time curves of the free base N-[5-(aminosulfonyl)-4-methyl-1,3-thiazol-2-yl]Nmethyl-2-[4-(2-pyridinyl)phenyl]acetamide at day 1 (after the first administration) were very similar in their general shape and slope to those profiles obtained in the single dose escalation trial (see Example a). As for the single dose escalation trial presented in Example a, there were dose-proportional increases in AUC₀₋₂₄ₕ and Cₘₐₓ at day 1.

During the 20-day treatment with administrations of the mono mesylate monohydrate salt of N-[5-(aminosulfonyl)-4-methyl-1,3-thiazol-2-yl]-N-methyl-2-[4-(2-pyridinyl)phenyl]-acetamide, wherein the particle size distribution is preferably defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g, once daily, the attainment of steady-state conditions was demonstrated by virtually identical minimal or “trough” concentrations of the free base of N-[5-(aminosulfonyl)-4-methyl-1,3-thiazol-2-yl]-N-methyl-2-[4-(2-pyridinyl)phenyl]acetamide achieved between days 9 and 13. At steady state, there was a low inter-individual variability of minimal or “trough” concentrations with CVs (coefficient of variations) between 16.7 and 21.7% (day 21). For all doses, the individual and mean concentration-time curves of the free base of N-[5-(aminosulfonyl)-4-methyl-1,3-thiazol-2-yl]-N-methyl-2-[4-(2-pyridinyl)phenyl]acet-amide at day 21 were very similar in their shape and slope to those profiles obtained at day 1.

Table c summarizes the steady-state pharmacokinetics of the free base of N-[5-(aminosulfonyl)-4-methyl-1,3-thiazol-2-yl]-N-methyl-2-[4-(2-pyridinyl)phenyl]acetamide.

Table c: Steady state pharmacokinetic parameters of the free base N-[5-(aminosulfonyl)-4-methyl-1,3-thiazol-2-yl]-N-methyl-2-[4-(2-pyridinyl)phenyl]acetamide at day 21 after daily administrations of 5, 25, or 100 mg as free base equivalent of the mono mesylate monohydrate salt of N-[5-(aminosulfonyl)-4-methyl-1,3-thiazol-2-yl]-N-methyl-2-[4-(2-pyridinyl)phenyl]acetamide in formulations in accordance with table a, wherein the particle size distribution is preferably defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g, to healthy volunteers (n=12 per dose).

In the following the abbreviations used in Table c are defined.

C_{trough}: measured plasma concentration immediately before dosing at day 21 (at the end of the dosing interval at steady state); AUC_{τ}, i.e. the steady state AUC (area under the curve) within the dosing interval of 24 hours, C_{max,ss} refers to the maximal observed analyte concentration at steady state. Cₐᵥ is defined as average plasma concentration during the dosing interval the mono mesylate monohydrate salt of N-[5-(aminosulfonyl)-4-methyl-1,3-thiazol-2-yl]-N-methyl-2-[4-(2-pyridinyl)phenyl]acetamide at steady state, R-AUC refers to the accumulation ratio of the AUC, i.e. AUC_{τ}/AUC_{0-24h,day1}, R-Cₘₐₓ refers to accumulation ratio of Cₘₐₓ, i.e. C_{max,ss}/C_{max,day1}; t_{1/2z} is defined as above

TABLE c

| dose [mg] | | | | | | | |
|---|---|---|---|---|---|---|---|
| as free | | AUC_{τ} | | | | | |
| base | C_{trough} | [ng · | C_{max,ss} | Cₐᵥ | | | t_{1/2z} |
| equivalent | [ng/mL] | h/mL] | [ng/mL] | [ng/mL] | R-AUC | R-Cₘₐₓ | [h] |
| 5 | 187 | 5094 | 301 | 213 | 5.2 | 5.0 | 82.6 |
| 25 | 832 | 23430 | 1358 | 977 | 5.3 | 5.3 | 68.6 |
| 100 | 3743 | 108800 | 6358 | 4540 | 5.1 | 5.3 | 59.8 |

For the three doses applied, there was a dose-proportional increase for all measures of exposure at steady state (C_{trough}, AUC_{τ}, C_{max,ss}, and Cₐᵥ) (see Table c).

For both AUC and Cₘₐₓ, the accumulation ratio R of all doses applied was very similar being approximately a factor of 5 (see Table c).

The time to reach C_{max,ss} was similar for the three doses (0.5-4.5 h). The peak-trough fluctuation at steady-state ranged between 59 and 64%.

Elimination half-life was in the same range as after single-dose application with 82.6 h (5 mg), 68.6 h (25 mg), and 59.8 h (100 mg). The apparent total clearance (CL/F) was estimated to be similar for all doses investigated (0.99-1.08 L/h).

Summary of the Results of Example c:

Under steady-state conditions, an increase in the dose of mono mesylate monohydrate salt of N-[5-(aminosulfonyl)-4-methyl-1,3-thiazol-2-yl]-N-methyl-2-[4-(2-pyridinyl)phenyl]acetamide in formulations in accordance with table a, wherein the particle size distribution is preferably defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g, resulted in a proportional increase in exposure to the resultant free base of N-[5-(aminosulfonyl)-4-methyl-1,3-thiazol-2-yl]-N-methyl-2-[4-(2-pyridinyl)phenyl]acetamide. In general, plasma concentrations at steady state are to be expected to be approximately five times higher than after single dose administration of the same dose. This should be a reflection of the half-life and dosing interval. Inter-individual variability of steady state exposure was quite low as revealed by a low coefficient of variation, e.g. for minimal or “trough” concentrations and peak-trough fluctuations. Rate of elimination and terminal half-lives at steady-state were comparable to the single dose situation.

### Example c

### Pharmacokinetic/Pharmacodynamic Correlation

To assess the pharmacokinetic/pharmacodynamic profile the effective dose of the specific free base of N-[5-(aminosulfonyl)-4-methyl-1,3-thiazol-2-yl]-N-methyl-2-[4-(2-pyridinyl)phenyl]-acetamide was validated in a murine HSV skin infection model and associated plasma concentrations were determined (data not shown).

The results were compared with the effective concentration of the free base of N-[5-(aminosulfonyl)-4-methyl-1,3-thiazol-2-yl]-N-methyl-2-[4-(2-pyridinyl)phenyl]acetamide in cell culture and correlated to exposures reached in healthy male volunteers in single and multiple dose phase I trials (see FIGS. 4-7).

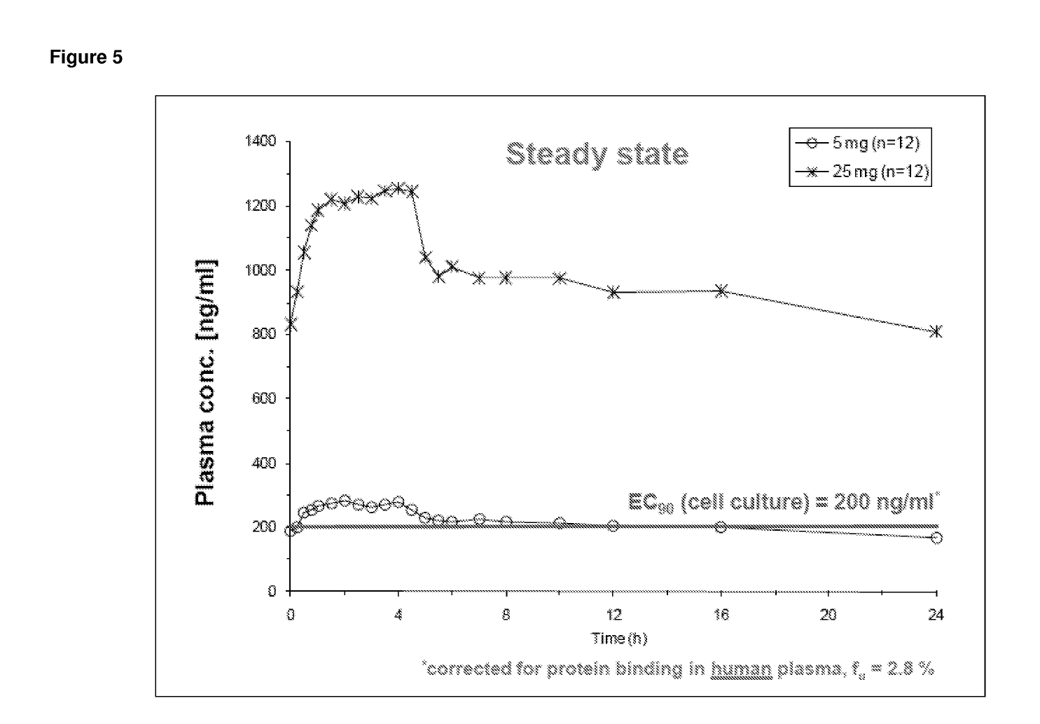

Oral doses of 5 mg/kg of the free base of N-[5-(aminosulfonyl)-4-methyl-1,3-thiazol-2-yl]-N-methyl-2-[4-(2-pyridinyl)phenyl]acetamide or higher once daily doses for four days completely suppressed the murine infection (data not shown). Associated plasma concentrations in mice determined with a single oral dose of 10 mg/kg of the free base form were well above the cell culture EC₉₀ adjusted for protein binding over the entire dosing interval of 24 h. In healthy male volunteers these plasma concentrations were covered by a single dose of 40 mg as free base equivalent to the crystalline mono mesylate monohydrate salt of N-[5-(aminosulfonyl)-4-methyl-1,3-thiazol-2-yl]-N-methyl-2-[4-(2-pyridinyl)phenyl]-acetamide (see FIG. 4) and at steady state by daily doses of 25 mg as free base equivalent (see FIG. 5) for 21 days. In both settings the resultant free base of N-[5-(aminosulfonyl)-4-methyl-1,3-thiazol-2-yl]-N-methyl-2-[4-(2-pyridinyl)phenyl]acetamide was safe and well tolerated up to the highest dose tested.

In summary, the free base of N-[5-(aminosulfonyl)-4-methyl-1,3-thiazol-2-yl]-N-methyl-2-[4-(2-pyridinyl)phenyl]acetamide exhibits advantageous PK/PD profiles in non-clinical studies and exposures required to suppress HSV replication were reached in humans.

Specifically, the free base of N-[5-(aminosulfonyl)-4-methyl-1,3-thiazol-2-yl]-N-methyl-2-[4-(2-pyridinyl)phenyl]acetamide, resultant from the herein described crystalline mono mesylate monohydrate salt of N-[5-(aminosulfonyl)-4-methyl-1,3-thiazol-2-yl]-N-methyl-2-[4-(2-pyridinyl)phenyl]acetamide wherein the particle size distribution is preferably defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g, exhibits advantageous PK/PD profiles in non-clinical studies and exposures required to suppress HSV replication were reached in humans.

These results clearly demonstrate that using the specific mono mesylate monohydrate salt of N-[5-(aminosulfonyl)-4-methyl-1,3-thiazol-2-yl]-N-methyl-2-[4-(2-pyridinyl)phenyl]-acetamide, wherein the particle size distribution is preferably defined by d(0.1) from 2 to 100 μm, d(0.5) from 30 to 210 μm and d(0.9) from 70 to 400 μm with a specific surface area of the particles less than 1.0 m²/g, and more preferably defined by d(0.1) from 10 to 75 μm, d(0.5) from 100 to 175 μm, d(0.9) from 200 to 350 μm with a specific surface area of the particles less than 0.3 m²/g in the formulations as described above, a once daily dose (or even a less frequent administration) is sufficient for reaching an appropriate plasma concentration for the treatment of AD in a subject that is having HSV-1 infection and is having AD or is having HSV-1 infection and is suspected of having AD. In a further human trial it has been shown that the administration of a higher dose of 400 mg to 600 mg as free base equivalent and preferably about 500 mg as free base equivalent of the crystalline mono mesylate monohydrate salt of the present invention is also sufficient for reaching an appropriate plasma concentration for the treatment of AD in a subject that is having HSV-1 infection and is having AD or is having HSV-1 infection and is suspected of having AD.

1. A method of treating Alzheimer's disease, comprising administering to a subject in need thereof an effective amount of a helicase-primase inhibitor according to Formula (I) or a pharmaceutically acceptable derivative, salt, solvate or hydrate thereof 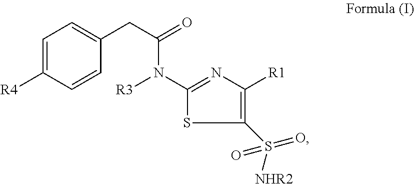
wherein
R1 is hydrogen, C1-C4 alkyl, or cycloalkyl,
R2 is hydrogen, C1-C4 alkyl, or cycloalkyl,
R3 is hydrogen, alkyl, cycloalkyl, heterocycloalkyl, haloalkyl, hydroxyalkyl, or alkoxyalkyl,
R4 is substituted or unsubstituted heteroaryl, or aryl, cycloalkyl denotes a non-aromatic ring system containing three to eight carbon atoms, wherein one or more of the carbon atoms in the ring are optionally substituted by a O, S, SO, SO₂, N or NR′,
R′ is independently H, haloalkyl, hydroxyalkyl, alkyl, cycloalkyl, aryl, or heteroaryl,
aryl denotes an aromatic group having five to ten carbon atoms, which is optionally substituted by one or more substituents R″;
R″ is independently H, —CO₂R′, —CONHR′, —CO-alkyl, —CN, alkyl, alkoxy, —OH, —SH, cycloalkyl, heterocycloalkyl, halogen, haloalkyl, haloalkyloxy, hydroxyalkyl, heteroaryl, or aryl, and
heteroaryl denotes a five- or six-membered heterocyclic group which contains at least one heteroatom selected from the group consisting of O, N, and S and which is optionally fused to another aromatic ring.

2. A method according to claim 1, wherein the subject has a herpes simplex virus type 1 infection and Alzheimer's disease or has an herpes simplex virus type 1 infection and is suspected of having Alzheimer's disease.

3. A method according to claim 2, wherein said subject has a herpes simplex virus type 1 infection and is suspected of having Alzheimer's disease, and said subject shows at least the below manifested symptoms of mild cognitive impairment during clinical examination:
a change in cognition
impairment in one or more cognitive domains
preservation of independence in functional abilities
not demented,
and wherein said subject is positive for herpes simplex virus type 1 infection when clinically examined by a herpes simplex virus test.

4. A method according to claim 2, wherein the subject is positive for a herpes simplex virus type 1 infection in an ex vivo herpes simplex virus test and possesses a specific genetic factor type 4 allele of the apolipoprotein E gene, which is APOE4, and said subject is positive for APOE4 in an ex vivo venous blood sample examined by an APOE genotyping test.

5. A method according to claim 2, wherein the subject is positive for a herpes simplex virus type 1 infection in an ex vivo herpes simplex virus test and said subject is positive for PSEN1 in an ex vivo PSEN1 test.

6. A method according to claim 2, wherein the subject is positive for a herpes simplex virus type 1 infection in an ex vivo herpes simplex virus test and said subject is positive for the presence of Aβ42 and P-tau in an ex vivo Tau/Aβ2 test.

7. A method according to claim 2, wherein the subject has a herpes simplex virus type 1 infection and Alzheimer's disease or has a herpes simplex virus type 1 infection and is suspected of having Alzheimer's disease,
wherein
R1 is hydrogen, or C1-C4 alkyl,
R2 is hydrogen, or C1-C4 alkyl,
R3 is hydrogen, alkyl, cycloalkyl, or heterocycloalkyl, and
R4 is substituted or unsubstituted heteroaryl, or aryl.

8. A method according to claim 2, wherein the subject has a herpes simplex virus type 1 infection and Alzheimer's disease or has a herpes simplex virus type 1 infection and is suspected of having Alzheimer's disease,
wherein
R1 is hydrogen,
R2 is hydrogen,
R3 is hydrogen, alkyl, or cycloalkyl, and
R4 is substituted or unsubstituted heteroaryl.

9. A method according to claim 2, wherein the compound of formula I is N-[5-(aminosulfonyl)-4-methyl-1,3-thiazol-2-yl]-N-methyl-2-[4-(2-pyridinyl)phenyl]acetamid 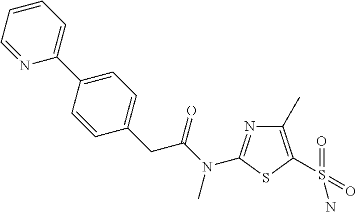 [SMILES: Cc1nc(N(C)C(=O)Cc2ccc(-c3ccccn3)cc2)sc1S(N)(=O)=O]
and wherein the subject has a herpes simplex virus type 1 infection and Alzheimer's disease or has a herpes simplex virus type 1 infection and is suspected of having Alzheimer's disease.

10. A method according to claim 2, wherein the compound of formula I is in a pharmaceutical composition comprising at least one pharmaceutically acceptable carrier, excipient, solvent and/or diluent.

11. A method according to claim 2, wherein the administration of the compound of formula I is by oral administration.

12. A method according to claim 2, wherein the compound of formula I is crystalline N-[5-(aminosulfonyl)-4-methyl-1,3-thiazol-2-yl]-N-methyl-2-[4-(2-pyridinyl)-phenyl]acetamide mono methanesulfonic acid monohydrate particles of the following formula 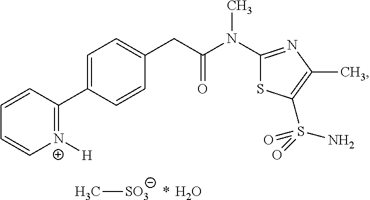
wherein said particles have a particle size range from 1 to 500 μm, a particle size distribution which is defined by d(0.1) from 2 to 100 μm, d(0.5) from 30 to 210 μm and d(0.9) from 70 to 400 μm and a specific surface area of less than 1.0 m²/g.

13. A method according to claim 12, wherein the N-[5-(aminosulfonyl)-4-methyl-1,3-thiazol-2-yl]-N-methyl-2-[4-(2-pyridinyl)-phenyl]acetamide mono methanesulfonic acid monohydrate particles have a particle size range from 2 μm to 400 μm.

14. A method according to claim 12, wherein the particles have a particle size distribution which is defined by d(0.1) from 10 to 75 μm, d(0.5) from 100 to 175 μm, and d(0.9) from 200 to 350 μm.

15. A method according to claim 12, wherein the particles have a specific surface area of less than 0.3 m²/g.

16. A method according to claim 12, wherein the crystalline N-[5-(aminosulfonyl)-4-methyl-1,3-thiazol-2-yl]-N-methyl-2-[4-(2-pyridinyl)phenyl]acetamide mono methanesulfonic acid monohydrate particles are in a pharmaceutical composition comprising at least one pharmaceutically acceptable carrier, excipient, solvent and/or diluent.

17. A method according to claim 16, wherein the crystalline N-[5-(aminosulfonyl)-4-methyl-1,3-thiazol-2-yl]-N-methyl-2-[4-(2-pyridinyl)-phenyl]acetamide mono methanesulfonic acid monohydrate particles have a particle size range from 2 μm to 400 μm.

18. A method according to claim 16, wherein the crystalline N-[5-(aminosulfonyl)-4-methyl-1,3-thiazol-2-yl]-N-methyl-2-[4-(2-pyridinyl)-phenyl]acetamide mono methanesulfonic acid monohydrate particles have a particle size distribution which is defined by d(0.1) from 10 to 75 μm, d(0.5) from 100 to 175 μm, and d(0.9) from 200 to 350 μm.

19. A method according to claim 16, wherein the crystalline N-[5-(aminosulfonyl)-4-methyl-1,3-thiazol-2-yl]-N-methyl-2-[4-(2-pyridinyl)-phenyl]acetamide mono methanesulfonic acid monohydrate particles have a specific surface area A method according to claim 16.

20. A method according to claim 16, wherein the free base of N-[5-(aminosulfonyl)-4-methyl-1,3-thiazol-2-yl]-N-methyl-2-[4-(2-pyridinyl)-phenyl]acetamide has an absolute bioavailability of 70%±30%, when administered in said composition containing at least 25 mg as free base equivalent of the crystalline N-[5-(aminosulfonyl)-4-methyl-1,3-thiazol-2-yl]-N-methyl-2-[4-(2-pyridinyl)-phenyl]acetamide mono methanesulfonic acid monohydrate to said subject that has a herpes simplex virus type 1 infection and Alzheimer's disease or has a herpes simplex virus type 1 infection and is suspected of having Alzheimer's disease.

21. A method according to claim 16, wherein the free base of N-[5-(aminosulfonyl)-4-methyl-1,3-thiazol-2-yl]-N-methyl-2-[4-(2-pyridinyl)-phenyl]acetamide in said subject has a mean maximum blood plasma concentration (mean Cₘₐₓ of at least one of
a) 608±184 ng/ml for a 40 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
b) 1306±125 ng/ml for a 80 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
c) 2613±1341 ng/ml for a 160 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
d) 3600±752 ng/ml for a 240 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
e) 4648±1813 ng/ml for a 320 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
f) 6926±1656 ng/ml for a 400 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered;
g) 6921±2190 ng/ml for a 480 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a single oral dose administered,
in said composition to said subject that has a herpes simplex virus type 1 infection and Alzheimer's disease or has a herpes simplex virus type 1 infection and is suspected of having Alzheimer's disease.

22. A method according to claim 16, wherein the free base of N-[5-(aminosulfonyl)-4-methyl-1,3-thiazol-2-yl]-N-methyl-2-[4-(2-pyridinyl)-phenyl]acetamide in a subject has a mean maximum blood plasma concentration (mean Cₘₐₓ) of at least one of
a) 608±184 ng/ml for a 40 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ₕ of 10090±3114 ng-h/ml in a subject for a 40 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 72±3 h on average; said dosage being a single oral dose administered;
b) 1306±125 ng/ml for a 80 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ₕ of 21940±2057 ng-h/ml in a subject for a 80 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 74±5 h on average; said dosage being a single oral dose administered;
c) 2613±1341 ng/ml for a 160 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ₕ of 40470±16700 ng-h/ml in a subject for a 160 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 63±6 h on average; said dosage being a single oral dose administered;
d) 3600±752 ng/ml for a 240 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ₕ of 59610±12770 ng-h/ml in a subject for a 240 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 64±5 h on average; said dosage being a single oral dose administered;
e) 4648±1813 ng/ml for a 320 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ₕ of 76250±27630 ng-h/ml in a subject for a 320 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 57±3 h on average; said dosage being a single oral dose administered;
f) 6926±1656 ng/ml for a 400 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ₕ of 104800±25740 ng-h/ml in a subject for a 400 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 57±4 h on average; said dosage being a single oral dose administered;
g) 6921±2190 ng/ml for a 480 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC₀₋₂₄ₕ of 112800±34260 ng-h/ml in a subject for a 480 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 53±4 h on average; said dosage being a single oral dose administered,
wherein t_{1/2z} is the elimination half-life,
in said composition to said subject that has a herpes simplex virus type 1 infection and Alzheimer's disease or has a herpes simplex virus type 1 infection and is suspected of having Alzheimer's disease.

23. A method according to claim 16, wherein free base of N-[5-(aminosulfonyl)-4-methyl-1,3-thiazol-2-yl]-N-methyl-2-[4-(2-pyridinyl)-phenyl]acetamide in a subject has a mean maximum blood plasma concentration at steady state (mean Cmax,ss) of at least one of
a) 1358±167 ng/ml for a 25 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a steady state dose after once daily single doses administered for 21 days;
b) 6358±1701 ng/ml for a 100 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a steady state dose after once daily single doses administered for 21 days;
c) 9987±2608 ng/ml for a 200 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, said dosage being a steady state dose after once daily single doses administered for 21 days,
in said composition to said subject that has a herpes simplex virus type 1 infection and Alzheimer's disease or has a herpes simplex virus type 1 infection and is suspected of having Alzheimer's disease.

24. A method according to claim 16, wherein the free base of N-[5-(aminosulfonyl)-4-methyl-1,3-thiazol-2-yl]-N-methyl-2-[4-(2-pyridinyl)-phenyl]acetamide in a subject has a mean maximum blood plasma concentration at steady state (mean C_{max,ss}) of at least one of
a) 1358±167 ng/ml for a 25 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC_{τ,ss} of 23430±3020 ng-h/ml in a subject for a 25 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 69±6 h on average, said dosage being a steady state dose after once daily single doses administered for 21 days;
b) 6358±1701 ng/ml for a 100 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate and/or an AUC_{τ,ss} of 108800±28610 ng-h/ml in a subject for a 100 mg dosage as free base equivalent of crystalline N-[5-(aminosulfonyl)-4-methyl-1,3-thiazol-2-yl]-N-methyl-2-[4-(2-pyridinyl)-phenyl]acetamide mono methanesulfonic acid monohydrate, and wherein t_{1/2z} is 60±4 h on average, said dosage being a steady state dose after once daily single doses administered for 21 days,
wherein t_{1/2z} is the elimination half-life,
in said composition to said subject that has a herpes simplex virus type 1 infection and Alzheimer's disease or has a herpes simplex virus type 1 infection and is suspected of having Alzheimer's disease.

25. A method according to claim 20, wherein said absolute bioavailability is achieved in a human.

26. A method according to claim 21, wherein said mean Cₘₐₓ is achieved in a human.

27. A method according to claim 22, wherein said AUC₀₋₂₄ₕ and t_{1/2z} is achieved in a human.

28. A method according to claim 24, wherein said AUC_{τ,ss} and t_{1/2z} is achieved in a human.

29. A method according to claim 23, wherein said mean C_{max,ss} is achieved in a human.

30. A method according to claim 1, wherein a compound of formula I or a pharmaceutically acceptable salt thereof is administered.
